(19) United States
(12) Patent Application Publication
(10) Pub. No.: US 2016/0333404 A1
(43) Pub. Date: Nov. 17, 2016

(54) METHODS FOR TREATING HCV

(71) Applicant: AbbVie Inc., North Chicago, IL (US)

(72) Inventors: Barry M. Bernstein, Mequon, WI (US); Isabelle A. Gaultier, Libertyville, IL (US); Daniel E. Cohen, Wilmette, IL (US); Jeffrey F. Waring, Franklin, WI (US); Stephen J. Abel, Bristol, WI (US); Emily O. Dumas, Libertyville, IL (US)

(21) Appl. No.: 14/557,524

(22) Filed: Dec. 2, 2014

Related U.S. Application Data
(63) Continuation-in-part of application No. 14/048,995, filed on Oct. 8, 2013, now abandoned.
(60) Provisional application No. 61/711,367, filed on Oct. 9, 2012.
(60) Provisional application No. 61/858,938, filed on Jul. 26, 2013.
(60) Provisional application No. 61/911,274, filed on Dec. 3, 2013.

(51) Int. Cl. C12Q 1/68; A61K 31/497; A61K 31/513; A61K 31/7056

(52) U.S. Cl. CPC C12Q 1/6876; A61K 31/7056; A61K 31/497; A61K 31/513; C12Q 2600/106; C12Q 2600/178

(57) ABSTRACT

The present invention features therapies for the treatment of HCV comprising direct-acting antiviral agents. Preferably, the treatment is administered to an HCV-infected patient who has been tested to determine expression levels of microRNAs such as miR-122 or miR-21. In one aspect, the therapies comprise administering one or more direct acting antiviral agents and, optionally ribavirin, to a subject with HCV infection. For example, the therapies comprise administering to the subject effective amounts of therapeutic agent 1, therapeutic agent 2, an inhibitor of cytochrome P450 (e.g., ritonavir), and ribavirin.

### RELATED APPLICATIONS

This application is a continuation-in-part of U.S. application Ser. No. 14/048,995, filed on Oct. 8, 2013, which claims the priority of U.S. provisional application Ser. No. 61/711,367, filed on Oct. 9, 2012, and U.S. provisional application Ser. No. 61/858,938, filed on Jul. 26, 2013. This application also claims the priority of U.S. provisional application Ser. No. 61/911,274, filed on Dec. 3, 2013. Each of the above-mentioned applications is incorporated by reference in its entirety.

### FIELD OF THE INVENTION

The present invention relates to treatment for hepatitis C virus (HCV) using a direct-acting antiviral regimen.

### BACKGROUND OF THE INVENTION

The HCV is an RNA virus belonging to the Hepacivirus genus in the Flaviviridae family. The enveloped HCV virion contains a positive stranded RNA genome encoding all known virus-specific proteins in a single, uninterrupted, open reading frame. The open reading frame comprises approximately 9500 nucleotides and encodes a single large polyprotein of about 3000 amino acids. The polyprotein comprises a core protein, envelope proteins E1 and E2, a membrane bound protein p7, and the non-structural proteins NS2, NS3, NS4A, NS4B, NS5A and NS5B.

Chronic HCV infection is associated with progressive liver pathology, including cirrhosis and hepatocellular carcinoma. Chronic HCV infection is characterized by high inter-individual variability in terms of response to currently approved treatments. Chronic HCV infection may be treated by peginterferon-alpha (PEG-IFNs) in combination with ribavirin and with or without one of the recently approved linear protease inhibitors, VX-950 (telaprevir) or SCH503034 (boceprevir). Substantial limitations to efficacy and tolerability remain as many users suffer from side effects, and viral elimination from the body is often incomplete. Therefore, there is a need for new therapies to treat HCV infection and a need to identify patients who will benefit from particular therapeutic regimens.

MicroRNAs (miRNAs) are short, non-translated RNA species that have been detected in many plants and animals. MiRNAs are 18- to 24-nucleotides in length and typically 22 nucleotides in length. It is estimated that there are more than 1500 miRNAs in the human genome. MicroRNAs have been shown to be present and stable in numerous biological fluids, including plasma and serum. Certain miRNAs are known for their ability to bind to complementary sequences on target messenger RNA transcripts, resulting in translational repression or target degradation and gene silencing. In these instances, the miRNA has been found to interact with sequences within the 3′ noncoding region (NCR) of the target messenger RNA. Each miRNA can regulate up to hundreds of targets and miRNAs are predicted to regulate a third of protein coding genes.

Sarasin-Filipowicz et al (Nat. Med. 2009 January; 15(1) 31-3) measured microRNA-122 (miR-122) levels in the liver in patients before and after IFN treatment. Treatment non-responders had significantly decreased pretreatment liver levels of miR-122. After treatment, miR-122 liver levels were unchanged.

### BRIEF SUMMARY OF THE INVENTION

It was unexpectedly discovered that patients with an inadequate sustained response (e.g., relapse) following treatment with a direct-acting antiviral regimen had higher baseline miR-122 and miR-21 expression levels as compared to patients that achieved an adequate sustained response (e.g., SVR) following treatment with a direct-acting antiviral regimen. Thus, baseline miR-122 and/or miR-21 levels can be used as a biomarker for individuals that may relapse or otherwise inadequately respond to treatment with a direct-acting antiviral regimen. Such information can be used to guide therapy.

It was also unexpectedly discovered that patients treated with a polymerase inhibitor alone exhibit decreases in miR-122 expression levels. Thus, miR-122 levels can be used as a biomarker to assess the efficacy of a polymerase inhibitor in a patient. Moreover, miR-122 levels can be used as a biomarker to predict whether a patient being treated with a direct-acting antiviral regimen comprising a polymerase inhibitor will achieve SVR. Based on the assessment of miR-122 levels, patients can be treated selectively, by choice of polymerase inhibitor, for HCV infection.

At least one aspect of the present invention provides methods to treat a patient infected with Hepatitis C virus with a direct-acting antiviral regimen. The methods comprise administering the direct-acting antiviral regimen to the patient. In the present methods, prior to the administration of the direct-acting antiviral regimen a blood sample obtained from the patient has been tested to determine a baseline miR-122 level. In some embodiments, the baseline miR-122 level is equal to or less than a mean miR-122 level in a population of HCV patients. In some embodiments, the miR-122 level is significantly less than a mean miR-122 level in a population of HCV patients who fail to achieve a sustained virological response following treatment with the direct-acting antiviral regimen. In some embodiments, the blood sample is a plasma sample or a serum sample. In some embodiments, the direct-acting antiviral regimen comprises one or more direct acting antiviral agents (DAAs). In some embodiments, the methods further comprise administering an inhibitor of cytochrome P-450 (such as ritonavir) to the patient to improve the pharmacokinetics or bioavailability of one or more of the DAAs. Preferably, the components of the direct-acting antiviral regimen are administered in amounts effective to provide a sustained virological response (SVR) or achieve another desired measure of effectiveness in a patient. In some embodiments, the direct-acting antiviral regimen is effective to reduce miR-122 levels in the patient. In some embodiments, the methods further comprise monitoring miR-122 levels in the patient at and/or between week 2 and week 10 after commencing administration of the direct-acting antiviral regimen. As further discussed herein, in certain embodiments, the patient is infected with HCV genotype 1, HCV genotype 2, or HCV genotype 3.

At least one aspect of the present invention provides methods to treat a patient infected with Hepatitis C virus with a direct-acting antiviral regimen. The methods comprise administering the direct-acting antiviral regimen to the patient. In the present methods, prior to the administration of the direct-acting antiviral regimen a blood sample obtained from the patient has been tested to determine a baseline miR-122 level. In some embodiments, the baseline miR-122 level is equal to or less than a mean miR-122 level in a population of HCV patients. In some embodiments, the miR-122 level is significantly less than a mean miR-122 level in a population of HCV patients who fail to achieve a sustained virological response following treatment with the direct-acting antiviral regimen. In some embodiments, the blood sample is a plasma sample or a serum sample. In some embodiments, the direct-acting antiviral regimen comprises one or more direct acting antiviral agents (DAAs). In some embodiments, the methods further comprise administering an inhibitor of cytochrome P-450 (such as ritonavir) to the patient to improve the pharmacokinetics or bioavailability of one or more of the DAAs. Preferably, the components of the direct-acting antiviral regimen are administered in amounts effective to provide a sustained virological response (SVR) or achieve another desired measure of effectiveness in a patient. In some embodiments, the direct-acting antiviral regimen is effective to reduce miR-122 levels in the patient. In some embodiments, the direct-acting antiviral regimen comprises a polymerase inhibitor effective to reduce miR-122 levels in the patient. In some embodiments, the methods further comprise monitoring miR-122 levels in the patient at and/or between week 2 and week 10 after commencing administration of the direct-acting antiviral regimen. As further discussed herein, in certain embodiments, the patient is infected with HCV genotype 1, HCV genotype 2, or HCV genotype 3.

At least one aspect of the present invention provides methods to treat a patient infected with Hepatitis C virus with a direct-acting antiviral regimen. The methods comprise administering the direct-acting antiviral regimen to the patient. In the present methods, prior to the administration of the direct-acting antiviral regimen a blood sample obtained from the patient has been tested to determine a baseline miR-21 level. In some embodiments, the baseline miR-21 level is equal to or less than a mean miR-21 level in a population of HCV patients. In some embodiments, the miR-21 level is significantly less than a mean miR-21 level in a population of HCV patients who fail to achieve a sustained virological response following treatment with the direct-acting antiviral regimen. In some embodiments, the blood sample is a plasma sample or a serum sample. In some embodiments, the direct-acting antiviral regimen comprises one or more DAAs. In some embodiments, the methods further comprise administering an inhibitor of cytochrome P-450 (such as ritonavir) to the patient to improve the pharmacokinetics or bioavailability of one or more of the DAAs. Preferably, the components of the direct-acting antiviral regimen are administered in amounts effective to provide an SVR or achieve another desired measure of effectiveness in a patient. In some embodiments, the methods further comprise monitoring miR-21 levels in the patient at and/or between week 2 and week 10 after commencing administration of the direct-acting antiviral regimen. As further discussed herein, in certain embodiments, the patient is infected with HCV genotype 1, HCV genotype 2, or HCV genotype 3.

At least one aspect of the present invention provides methods to treat a patient infected with Hepatitis C virus with a direct-acting antiviral regimen. The methods comprise administering the direct-acting antiviral regimen to the patient. In the present methods, prior to the administration of the direct-acting antiviral regimen a sample obtained from the patient has been tested to determine a baseline miR-122 or miR-21 expression level. In some embodiments, the baseline miR-122 or miR-21 expression level is equal to or less than a pre-determined control level. In some embodiments, the baseline miR-122 or miR-21 expression level is at least 1-fold less than a pre-determined control level. In some embodiments, the baseline miR-122 or miR-21 expression level is at least 2-fold less than a pre-determined control level. In some embodiments, the baseline miR-122 or miR-21 expression level is at least 3-fold less than a pre-determined control level. In some embodiments, the baseline miR-122 or miR-21 expression level is at least 4-fold less than a pre-determined control level. In some embodiments, the baseline miR-122 or miR-21 expression level is from 1-fold to 4-fold less than a pre-determined control level. The pre-determined control level can be a range or a specific value. The pre-determined control level can be determined empirically, such as, by obtaining a mean expression level from a population of subjects. The population of subjects can be a population of healthy subjects or a population of HCV-infected subjects. Thus, the pre-determined control level can be, for example, a mean expression level from a population of patients infected with Hepatitis C virus. In some embodiments, the miR-122 or miR-21 expression level can be determined by measuring circulating miR-122 or miR-21 in plasma or serum samples obtained from the patient infected with Hepatitis C virus. In some embodiments, the direct-acting antiviral regimen comprises one or more DAAs. In some embodiments, the methods further comprise administering an inhibitor of cytochrome P-450 (such as ritonavir) to the patient to improve the pharmacokinetics or bioavailability of one or more of the DAAs. Preferably, the components of the direct-acting antiviral regimen are administered in amounts effective to provide an SVR or achieve another desired measure of effectiveness in a patient. In some embodiments, the direct-acting antiviral regimen is effective to reduce miR-122 or miR-21 expression levels in the patient. In some embodiments, the direct-acting antiviral regimen comprises a polymerase inhibitor effective to reduce miR-122 or miR-21 expression levels in the patient. As further discussed herein, in certain embodiments, the patient is infected with HCV genotype 1, HCV genotype 2, or HCV genotype 3.

Another aspect of the present invention provides methods to treat a patient having a Hepatitis C virus infection with a direct-acting antiviral regimen. The methods comprise administering the direct-acting antiviral regimen to the patient for a duration of 12 or more weeks. In the present methods, prior to the administration of the direct-acting antiviral regimen a sample obtained from the patient has been tested to determine a baseline miR-122 or miR-21 expression level. In some embodiments, the baseline miR-122 or miR-21 expression level is greater than a pre-determined control level. In some embodiments, the baseline miR-122 or miR-21 expression level is at least 1-fold greater than a pre-determined control level. In some embodiments, the baseline miR-122 or miR-21 expression level is at least 2-fold greater than a pre-determined control level. In some embodiments, the baseline miR-122 or miR-21 expression level is at least 3-fold greater than a pre-determined control level. In some embodiments, the baseline miR-122 or miR-21 expression level is at least 4-fold greater than a pre-determined control level. In some embodiments, the baseline miR-122 or miR-21 expression level is from 1-fold to 4-fold greater than a pre-determined control level. The pre-determined control level can be a range or a specific value. The pre-determined control level can be determined empirically, such as, by obtaining a mean expression level from a population of subjects. The population of subjects can be a population of healthy subjects or a population of HCV-infected subjects. Thus, the pre-determined control level can be, for example, a mean expression level from a population of patients infected with Hepatitis C virus. In some embodiments, the miR-122 or miR-21 expression level can be determined by measuring circulating miR-122 or miR-21 in plasma or serum samples obtained from the patient infected with Hepatitis C virus. In some embodiments, the direct-acting antiviral regimen comprises one or more DAAs. In some embodiments, the methods further comprise administering an inhibitor of cytochrome P-450 (such as ritonavir) to the patient to improve the pharmacokinetics or bioavailability of one or more of the DAAs. Preferably, the components of the direct-acting antiviral regimen are administered in amounts effective to provide an SVR or achieve another desired measure of effectiveness in a patient. In some embodiments the duration is 16, 20, or 24 weeks. In some embodiments, the direct-acting antiviral regimen is effective to reduce miR-122 or miR-21 expression levels in the patient. In some embodiments, the direct-acting antiviral regimen comprises a polymerase inhibitor effective to reduce miR-122 or miR-21 expression levels in the patient. As further discussed herein, in certain embodiments, the patient is infected with HCV genotype 1, HCV genotype 2, or HCV genotype 3.

Yet another aspect of the present invention provides methods to treat a patient having a Hepatitis C virus infection. The methods comprise administering a first direct-acting antiviral regimen to the patient; assessing microRNA expression in a sample obtained from the patient after the administration of the first direct-acting antiviral regimen to establish an on-treatment microRNA expression level; and administering a second direct-acting antiviral regimen to the patient when the on-treatment microRNA expression level is equal to a baseline expression microRNA level determined from a sample obtained from the patient prior to administration of the first direct-acting antiviral regimen. The microRNA can be miR-122 or miR-21. In some embodiments, the miR-122 or miR-21 expression level can be determined by measuring circulating miR-122 or miR-21 in plasma or serum samples obtained from the patient infected with Hepatitis C virus. In some embodiments, the first direct-acting antiviral regimen comprises one or more DAAs. In some embodiments, the second direct-acting antiviral regimen comprises one or more DAAs. In some embodiments, the methods further comprise co-administering an inhibitor of cytochrome P-450 (such as ritonavir) with the first or second direct-acting antiviral regimen. Preferably, the components of the first or second direct-acting antiviral regimen are administered in amounts effective to provide an SVR or achieve another desired measure of effectiveness in a patient. In some embodiments, the direct-acting antiviral regimen is effective to reduce miR-122 or miR-21 expression levels in the patient. In some embodiments, the direct-acting antiviral regimen comprises a polymerase inhibitor effective to reduce miR-122 or miR-21 expression levels in the patient. As further discussed herein, in certain embodiments, the patient is infected with HCV genotype 1, HCV genotype 2, or HCV genotype 3.

Another aspect of the present invention provides methods to treat a patient infected with Hepatitis C virus. The methods comprise identifying a patient having an miR-122 or miR-21 expression level that is predictive of a sustained response to a direct-acting antiviral regimen; selecting the identified patient for treatment with the direct-acting antiviral regimen; and administering the direct-acting antiviral regimen to the selected patient. In some embodiments, the miR-122 or miR-21 expression level in the patient is equal to or less than a pre-determined control level. In some embodiments, the miR-122 or miR-21 expression level in the identified patient can be at least four-fold less than a pre-determined control level. In some embodiments, the pre-determined control level is a mean expression level from a population of patients infected with Hepatitis C virus. In some embodiments, the miR-122 or miR-21 expression level is determined by measuring circulating miR-122 or miR-21 in plasma or serum samples obtained from the patient infected with Hepatitis C virus. In some embodiments, the direct-acting antiviral regimen comprises one or more DAAs. In some embodiments, the methods further comprise administering an inhibitor of cytochrome P-450 (such as ritonavir) to the patient to improve the pharmacokinetics or bioavailability of one or more of the DAAs. In some embodiments, the direct-acting antiviral regimen comprises an HCV protease inhibitor and an HCV polymerase inhibitor. Preferably, the components of the direct-acting antiviral regimen are administered in amounts effective to provide an SVR or achieve another desired measure of effectiveness in a subject. In some embodiments, the direct-acting antiviral regimen is effective to reduce miR-122 or miR-21 expression levels in the patient. In some embodiments, the direct-acting antiviral regimen comprises a polymerase inhibitor effective to reduce miR-122 or miR-21 expression levels in the patient.

Yet another aspect of the present invention provides methods to treat a patient having a Hepatitis C virus infection with a direct-acting antiviral regimen. The methods comprise assessing miR-122 or miR-21 expression in a sample obtained from a patient infected with Hepatitis C virus to obtain a microRNA expression level; predicting responsiveness to the direct-acting antiviral regimen, wherein microRNA expression level that is less than a pre-determined control level is predictive of a sustained response to the direct-acting antiviral regimen; and administering the direct-acting antiviral regimen to the patient based upon the microRNA expression level. In some embodiments, the pre-determined control level is a mean expression level from a population of patients infected with Hepatitis C virus. In some embodiments, the sample is a plasma or serum sample. In some embodiments, the direct-acting antiviral regimen comprises at least two DAAs, with or without ribavirin. In some embodiments, the methods further comprise administering an inhibitor of cytochrome P-450 (such as ritonavir) to the patient to improve the pharmacokinetics or bioavailability of one or more of the DAAs. In some embodiments, the direct-acting antiviral regimen comprises an HCV protease inhibitor and an HCV polymerase inhibitor. Preferably, the components of the direct-acting antiviral regimen are administered in amounts effective to provide an SVR or achieve another desired measure of effectiveness in a patient. In some embodiments, the direct-acting antiviral regimen is effective to reduce miR-122 or miR-21 expression levels in the patient. In some embodiments, the direct-acting antiviral regimen comprises a polymerase inhibitor effective to reduce miR-122 or miR-21 expression levels in the patient.

As another aspect, methods for treating HCV infection in a patient who has been tested to establish a baseline miR-122 or miR-21 expression level for the patient are provided. The methods comprise administering a protease inhibitor and one or more polymerase inhibitors to the patient. The protease inhibitor can be therapeutic agent 1. The polymerase inhibitor can be therapeutic agent 2. The methods also can comprise administering ribavirin and/or an inhibitor of cytochrome P-450 to the patient. Preferably, therapeutic agent 1, the polymerase inhibitor(s), ribavirin and the inhibitor of cytochrome P-450 are administered in amounts effective to provide SVR or another measure of effectiveness in the patient. As non-limiting examples, therapeutic agent 1 and the inhibitor of cytochrome P-450 can be co-formulated and administered once daily, and the polymerase inhibitor(s) can be administered twice daily.

Yet another aspect of the present invention provides methods to treat a patient having a Hepatitis C virus infection with a direct-acting antiviral regimen that includes a dose of a polymerase inhibitor (a “polymerase inhibitor-containing regimen”). The methods comprise assessing miR-122 expression in a sample obtained from a patient infected with Hepatitis C virus to obtain a baseline miR-122 expression level; administering the polymerase inhibitor-containing regimen to the patient; assessing miR-122 expression in a sample obtained from the patient after the administration of the polymerase inhibitor-containing regimen to establish an on-treatment miR-122 expression level; and increasing the dose of the polymerase inhibitor and/or administering a second direct-acting antiviral regimen to the patient when the on-treatment miR-122 expression level is not substantially different from the baseline miR-122 expression level. In some embodiments, the dose of the polymerase inhibitor is adjusted based on the on-treatment miR-122 expression level. In some embodiments, the dose of the polymerase inhibitor is adjusted to be effective to reduce miR-122 expression levels. In some embodiments, the second direct-acting antiviral regimen is effective to reduce miR-122 expression levels in the patient. In some embodiments, the second direct-acting antiviral regimen comprises a second polymerase inhibitor that is effective to reduce miR-122 expression levels in the patient. In some embodiments, the dose of the second polymerase inhibitor in the direct-acting antiviral regimen is effective to reduce miR-122 expression levels in the patient. In some embodiments, the miR-122 expression level can be determined by measuring circulating miR-122 in plasma or serum samples obtained from the patient infected with Hepatitis C virus. In some embodiments, the polymerase inhibitor-containing regimen comprises one or more additional DAAs. In some embodiments, the second direct-acting antiviral regimen comprises one or more DAAs. In some embodiments, the methods further comprise co-administering an inhibitor of cytochrome P-450 (such as ritonavir) with the first or second direct-acting antiviral regimen. Preferably, the components of the first or second direct-acting antiviral regimen are administered in amounts effective to provide an SVR or achieve another desired measure of effectiveness in a patient.

Yet another aspect of the present invention provides methods to treat a patient having a Hepatitis C virus infection with a direct-acting antiviral regimen, wherein the direct-acting antiviral regimen comprises a dose of a polymerase inhibitor (a “polymerase inhibitor-containing regimen”). The methods comprise assessing miR-122 expression in a sample obtained from a patient infected with Hepatitis C virus to obtain a miR-122 expression level; predicting responsiveness to the polymerase inhibitor-containing direct-acting antiviral regimen, wherein miR-122 expression level that is less than or equal to a pre-determined control level is predictive of a sustained response to the polymerase inhibitor-containing direct-acting antiviral regimen; and continuing to administer the polymerase inhibitor-containing direct-acting antiviral regimen to the patient based upon the miR-122 expression level. If the miR-122 expression level is above the pre-determined control level, the method can include increasing the dose of the polymerase inhibitor and/or administering a second polymerase inhibitor. In some embodiments, the pre-determined control level is a mean expression level from a population of patients infected with Hepatitis C virus. In some embodiments, the sample is a plasma or serum sample. In some embodiments, the polymerase inhibitor-containing direct-acting antiviral regimen comprises at least one additional DAA, with or without ribavirin. In some embodiments, the methods further comprise administering an inhibitor of cytochrome P-450 (such as ritonavir) to the patient to improve the pharmacokinetics or bioavailability of one or more of the DAAs. In some embodiments, the direct-acting antiviral regimen comprises an HCV protease inhibitor and an HCV polymerase inhibitor. Preferably, the components of the direct-acting antiviral regimen are administered in amounts effective to provide an SVR or achieve another desired measure of effectiveness in a patient. In some embodiments, the direct-acting antiviral regimen is effective to reduce miR-122 expression levels in the patient. In some embodiments, the dose of the polymerase inhibitor in the direct-acting antiviral regimen is effective to reduce miR-122 expression levels in the patient. In some embodiments, the dose of the polymerase inhibitor is adjusted based on miR-122 expression levels.

Yet another aspect of the present invention provides methods to treat a patient having a Hepatitis C virus infection. The methods comprise administering a first direct-acting antiviral regimen to the patient, wherein the first direct-acting antiviral regimen comprises a first polymerase inhibitor; assessing miR-122 expression in a sample obtained from the patient after the administration of the first direct-acting antiviral regimen to establish an on-treatment miR-122 expression level; and administering a second direct-acting antiviral regimen to the patient when the on-treatment miR-122 expression level is equal to or greater than a baseline miR-122 expression level determined from a sample obtained from the patient prior to administration of the first direct-acting antiviral regimen. In some embodiments, the miR-122 expression level is determined by measuring circulating miR-122 in plasma or serum samples obtained from the patient infected with Hepatitis C virus. In some embodiments, the miR-122 expression is assessed using real time polymerase chain reaction. In some embodiments, the sample obtained from the patient after the administration of the first direct-acting antiviral regimen is a serum or plasma sample. In some embodiments, the second direct-acting antiviral regimen comprises a higher dose of the first polymerase inhibitor of the first direct-acting antiviral regimen, a second polymerase inhibitor, or both.

Yet another aspect of the present invention provides methods to treat a patient having a Hepatitis C virus infection. The methods comprise administering a first direct-acting antiviral regimen to the patient, wherein the first direct-acting antiviral regimen comprises a first polymerase inhibitor; requesting results of one or more tests that provide a baseline microRNA expression level and an on-treatment microRNA expression level; and administering a second direct-acting antiviral regimen to the patient when the on-treatment microRNA expression level is not substantially different from the baseline microRNA expression level. In some embodiments, the microRNA is miR-122. In some embodiments, the microRNA is miR-21. In some embodiments, the method comprises administering a second direct-acting antiviral regimen to the patient when the on-treatment microRNA expression level is equal to or greater than the baseline microRNA expression level. In some embodiments, the one or more tests assess circulating microRNA in plasma or serum samples obtained from the patient. In some embodiments, the circulating microRNA is assessed using real time polymerase chain reaction. In some embodiments, the second direct-acting antiviral regimen comprises a higher dose of the first polymerase inhibitor of the first direct-acting antiviral regimen, a second polymerase inhibitor, or both. In some embodiments, the dose of the first polymerase inhibitor is adjusted based on the on-treatment miR-122 expression level. In some embodiments, the second direct-acting antiviral regimen comprises at least two DAAs. Each DAA can be selected from, for example, HCV protease inhibitors, HCV polymerase inhibitors, or HCV NS5A inhibitors. In some embodiments, the patient is infected with HCV genotype 1, HCV genotype 2, or HCV genotype 3.

Yet another aspect of the present invention provides methods to predict responsiveness of an HCV-infected patient to a direct-acting antiviral regimen. The direct-acting antiviral regimen may include a polymerase inhibitor. The methods comprise assessing miR-122 expression in a sample obtained from a patient infected with Hepatitis C virus to obtain a miR-122 expression level; and predicting responsiveness to the direct-acting antiviral regimen, wherein miR-122 expression level that is less than or equal to a pre-determined control level is predictive of a sustained response to the direct-acting antiviral regimen. In some embodiments, the pre-determined control level is a mean expression level from a population of patients infected with Hepatitis C virus. In some embodiments, the sample is a plasma or serum sample. In some embodiments, the polymerase inhibitor-containing direct-acting antiviral regimen comprises at least one additional DAA, with or without ribavirin. In some embodiments, the methods further comprise administering an inhibitor of cytochrome P-450 (such as ritonavir) to the patient to improve the pharmacokinetics or bioavailability of one or more of the DAAs. In some embodiments, the direct-acting antiviral regimen comprises an HCV protease inhibitor and an HCV polymerase inhibitor. Preferably, the components of the direct-acting antiviral regimen are administered in amounts effective to provide an SVR or achieve another desired measure of effectiveness in a patient. In some embodiments, the direct-acting antiviral regimen is effective to reduce miR-122 expression levels in the patient.

Yet another aspect of the present invention provides methods to predict responsiveness of an HCV-infected patient to a direct-acting antiviral regimen. In some embodiments, the direct-acting antiviral regimen comprises a polymerase inhibitor. In some embodiments, the polymerase inhibitor-containing direct-acting antiviral regimen comprises at least one additional DAA, with or without ribavirin. In some embodiments, the direct-acting antiviral regimen comprises an HCV protease inhibitor and an HCV polymerase inhibitor. The methods comprise assessing microRNA expression in a sample obtained from a patient infected with Hepatitis C virus to obtain a microRNA expression level; and predicting responsiveness to the direct-acting antiviral regimen. In certain embodiments, a microRNA expression level that is less than or equal to a pre-determined control level is predictive of a sustained response to the direct-acting antiviral regimen. In some embodiments, a microRNA expression level that is greater than a pre-determined control level predicts an inadequate sustained response to treatment with the direct-acting antiviral regimen. The pre-determined control level can be a range or a specific value. The pre-determined control level can be determined empirically, such as, by obtaining a mean expression level from a population of subjects. The population of subjects can be a population of healthy subjects or a population of HCV-infected subjects. In some embodiments, the pre-determined control level is a mean expression level from a population of patients infected with Hepatitis C virus. In some embodiments, the sample is a plasma or serum sample. In some embodiments, the step of assessing microRNA expression comprises hybridizing a nucleic acid primer or probe to the microRNA or a complementary sequence thereof to form a detectable complex. In some embodiments, the step of assessing microRNA expression comprises generating an amplicon by polymerase chain reaction. In some embodiments, the step of assessing microRNA expression comprises amplifying all or part of the microRNA or complementary sequence thereof. In some embodiments, the step of assessing microRNA expression comprises generating cDNA from an RNA-containing sample and sequencing at least a portion of the cDNA.

In the foregoing methods as well as methods described herein, the methods can include administering ribavirin. The methods also can include administering ritonavir or another CYP3A4 inhibitor (e.g., cobicistat) if one of the DAAs requires or benefits from pharmacokinetic enhancement. Where the direct-acting antiviral regimen comprises at least two DAAs, the at least two DAAs can be administered concurrently or sequentially. For example, one DAA can be administered once daily, and another DAA can be administered twice daily. As a non-limiting example, the patient being treated can be infected with HCV genotype 1, such as genotype 1a or 1b. As another non-limiting example, the patient can be infected with HCV genotype 2 or 3. As yet another non-limiting example, the patient can be a HCV-treatment naïve patient, a HCV-treatment experienced patient, an interferon non-responder (e.g., a null responder, a partial responder or a relapser), or not a candidate for interferon treatment.

In the foregoing methods as well as methods described hereinbelow, the direct-acting antiviral regimen can comprise protease inhibitors, nucleoside or nucleotide polymerase inhibitors, non-nucleoside polymerase inhibitors, NS3B inhibitors, NS4A inhibitors, NS5A inhibitors, NS5B inhibitors, cyclophilin inhibitors, and combinations of any of the foregoing. For example, in some embodiments, the direct-acting antiviral regimen can comprise or consist of at least one HCV protease inhibitor and at least one HCV polymerase inhibitor. The HCV polymerase inhibitor can be a nucleotide or nucleoside polymerase inhibitor or a non-nucleoside polymerase inhibitor. The HCV polymerase inhibitor can also be a non-nucleotide polymerase inhibitor.

In the foregoing methods as well as methods described hereinbelow, the DAAs can be selected from the group consisting of protease inhibitors, nucleoside or nucleotide polymerase inhibitors, non-nucleoside polymerase inhibitors, NS3B inhibitors, NS4A inhibitors, NS5A inhibitors, NS5B inhibitors, cyclophilin inhibitors, and combinations of any of the foregoing.

For example, in some embodiments, the DAAs used in the present methods comprise or consist of at least one HCV protease inhibitor and at least one HCV polymerase inhibitor. The HCV polymerase inhibitor can be a nucleotide or nucleoside polymerase inhibitor or a non-nucleoside polymerase inhibitor. The HCV polymerase inhibitor can also be a non-nucleotide polymerase inhibitor. In some embodiments, the HCV protease inhibitor is therapeutic agent 1 (described below) and the HCV polymerase inhibitor is therapeutic agent 2 (also described below). By way of example, therapeutic agent 1 can be administered at a total daily dose of 100 mg, alternatively 200 mg, or alternatively 250 mg. By way of example, therapeutic agent 1 can be administered in a total daily dose of from 100 mg to 250 mg, or administered at least once daily at a dose of from 150 mg to 250 mg, and therapeutic agent 2 can be administered twice daily at doses from 200 mg to 400 mg. For some embodiments, the HCV protease inhibitor is therapeutic agent 1 and the HCV polymerase inhibitor is a non-nucleos/tide polymerase inhibitor. Ritonavir (or another cytochrome P-450 3A4 inhibitor) can be co-administered with therapeutic agent 1 to improve the pharmacokinetics and bioavailability of therapeutic agent 1.

In some embodiments, the DAAs used in the present methods comprise or consist of at least one HCV protease inhibitor, at least one HCV polymerase inhibitor, and at least one NS5A inhibitor. The HCV polymerase inhibitor can be a nucleotide or nucleoside polymerase inhibitor or a non-nucleoside polymerase inhibitor. The HCV polymerase inhibitor can also be a non-nucleotide polymerase inhibitor. In some embodiments, the HCV protease inhibitor is therapeutic agent 1 (described below), the HCV polymerase inhibitor is therapeutic agent 2 (also described below), and the NS5A inhibitor is therapeutic agent 3 (also described below). By way of example, therapeutic agent 3 may be administered in a total daily dose amount of from 5 mg to 300 mg, or from 25 mg to 200 mg, or from 25 mg to 50 mg or any amounts there between. In some embodiments, the total daily dosage amount for therapeutic agent 3 is 25 mg.

In the foregoing methods as well as methods described herein, a DAA can be administered in any effective dosing schemes and/or frequencies, for example, each DAA can be administered daily. Each DAA can be administered either separately or in combination, and each DAA can be administered at least once a day, at least twice a day, or at least three times a day. Likewise, the ribavirin can be administered at least once a day, at least twice a day, or at least three times a day, either separately or in combination with one or more of the DAAs. In some preferred embodiments, therapeutic agent 1 is administered once daily. In some preferred embodiments, therapeutic agent 2 is administered twice daily.

In some aspects, the direct-acting antiviral regimen can comprise (i) Compound 1 or a pharmaceutically acceptable salt thereof, which is co-administered or co-formulated with ritonavir, and (ii) Compound 2 or a pharmaceutically acceptable salt thereof. In some embodiments, the regimen can also comprise Compound 3 or a pharmaceutically acceptable salt thereof.

In yet another aspect, the direct-acting antiviral regimen can comprise a drug or drug combination selected from the group consisting of: GS-7977, a combination of GS-7977 and GS-5885, a combination of GS-7977 and GS-9669, a combination of BMS-790052 (daclatasvir) and BMS-650032 (asunaprevir), a combination of BI-201335 and BI-27127, a combination of BMS-986094 and BMS-790052 (daclatasvir), a combination of GS-7977 and PSI-938, a combination of GS-5885 and GS-9451, a combination of GS-5885, GS-9190 and GS-9451, a combination of telaprevir and VX-222, a combination of telaprevir and ALS-2200, a combination of danoprevir and R05466731 (setrobuvir), a combination of danoprevir, RO5466731 (setrobuvir), and R7128 (mericitabine), and a combination of danoprevir and R7128 (mericitabine). In yet other aspects, the direct-acting antiviral regimen can comprise a combination of GS-7977 and BMS-790052 (daclatasvir), a combination of GS-7977 and TMC-435 (simeprevir), a combination of TMC-435 (simeprevir) and TMC647055, or a combination of BMS-790052 (daclatasvir) and TMC-435 (simeprevir). In yet another aspect, the at least two direct acting antiviral agents comprises a combination of GS-7977 and BMS-650032 (asunaprevir). In still another aspect, the at least two direct acting antiviral agents comprises a combination of GS-7977, BMS-650032 (asunaprevir) and BMS-790052 (daclatasvir).

In another aspect, the present technology provides a combination of Compound 1 (or a pharmaceutically acceptable salt thereof) and Compound 2 (or a pharmaceutically acceptable salt thereof) for use in treating HCV infection. The treatment comprises administering the DAAs to an HCV-infected patient who has undergone testing to determine the patient's miR-122 expression level in blood prior to the treatment. The treatment includes administering ribavirin but does not include administering interferon; and ritonavir or another CYP3A4 inhibitor (e.g., cobicistat) is administered with Compound 1 (or the salt thereof) to improve the pharmacokinetics of the latter. Preferably, the treatment lasts 12 weeks, and the patient's pre-treatment miR-122 level in blood is significantly lower than a mean pre-treatment miR-122 blood level in HCV patients who rebound after the 12-week treatment. Also preferably, the treatment lasts 12 weeks, and the patient's pre-treatment miR-122 level in blood is at least 1-fold lower than a mean pre-treatment miR-122 blood level in HCV patients who rebound after the 12-week treatment. Also preferably, the treatment lasts 12 weeks, and the patient's pre-treatment miR-122 level in blood is at least 2-fold lower than a mean pre-treatment miR-122 blood level in HCV patients who rebound after the 12-week treatment. Also preferably, the treatment lasts 12 weeks, and the patient's pre-treatment miR-122 level in blood is at least 3-fold lower than a mean pre-treatment miR-122 blood level in HCV patients who rebound after the 12-week treatment. Also preferably, the treatment lasts 12 weeks, and the patient's pre-treatment miR-122 level in blood is at least 4-fold lower than a mean pre-treatment miR-122 blood level in HCV patients who rebound after the 12-week treatment. Also preferably, the treatment lasts 12 weeks, and the patient's pre-treatment miR-122 level in blood is from 1-fold to 4-fold lower than a mean pre-treatment miR-122 blood level in HCV patients who rebound after the 12-week treatment. Compound 1 (or the salt thereof) and Compound 2 (or the salt thereof) can be administered concurrently or sequentially. For example, Compound 1 (or the salt thereof) can be administered once daily, together with ritonavir or another CYP3A4 inhibitor (e.g., cobicistat), and Compound 2 (or the salt thereof) can be administered twice daily. For yet another example, Compound 1 (or the salt thereof) and ritonavir (or another CYP3A4 inhibitor, e.g., cobicistat) are co-formulated in a single composition and administered concurrently (e.g., once daily). For yet another example, Compound 1 (or the salt thereof), co-formulated with ritonavir (or another CYP3A4 inhibitor, e.g., cobicistat), is administered once daily, and Compound 2 (or the salt thereof) is administered twice daily. As a non-limiting example, the patient being treated can be infected with HCV genotype 1, such as genotype la or lb. As another non-limiting example, the patient can be infected with HCV genotype 2 or 3. As yet another non-limiting example, the patient can be a HCV-treatment naïve patient, a HCV-treatment experienced patient, an interferon non-responder (e.g., a null responder), or not a candidate for interferon treatment. The present technology also features the same aspect of the invention as described immediately above, except that the treatment does not include administering either ribavirin or interferon.

In another aspect, the present technology provides a combination of Compound 1 (or a pharmaceutically acceptable salt thereof) and Compound 2 (or a pharmaceutically acceptable salt thereof) for use in treating HCV infection. The treatment comprises administering the DAAs to an HCV-infected patient who has undergone testing to determine the patient's miR-21 expression level in blood prior to the treatment. The treatment includes administering ribavirin but does not include administering interferon; and ritonavir or another CYP3A4 inhibitor (e.g., cobicistat) is administered with Compound 1 (or the salt thereof) to improve the pharmacokinetics of the latter. Preferably, the treatment lasts 12 weeks, and the patient's pre-treatment miR-21 level in blood is significantly lower than a mean pre-treatment miR-21 blood level in HCV patients who rebound after the 12-week treatment. Also preferably, the treatment lasts 12 weeks, and the patient's pre-treatment miR-21 level in blood is at least 1-fold lower than a mean pre-treatment miR-21 blood level in HCV patients who rebound after the 12-week treatment. Also preferably, the treatment lasts 12 weeks, and the patient's pre-treatment miR-21 level in blood is at least 2-fold lower than a mean pre-treatment miR-21 blood level in HCV patients who rebound after the 12-week treatment. Also preferably, the treatment lasts 12 weeks, and the patient's pre-treatment miR-21 level in blood is at least 3-fold lower than a mean pre-treatment miR-21 blood level in HCV patients who rebound after the 12-week treatment. Also preferably, the treatment lasts 12 weeks, and the patient's pre-treatment miR-21 level in blood is at least 4-fold lower than a mean pre-treatment miR-21 blood level in HCV patients who rebound after the 12-week treatment. Also preferably, the treatment lasts 12 weeks, and the patient's pre-treatment miR-21 level in blood is from 1-fold to 4-fold lower than a mean pre-treatment miR-21 blood level in HCV patients who rebound after the 12-week treatment. Compound 1 (or the salt thereof) and Compound 2 (or the salt thereof) can be administered concurrently or sequentially. For example, Compound 1 (or the salt thereof) can be administered once daily, together with ritonavir or another CYP3A4 inhibitor (e.g., cobicistat), and Compound 2 (or the salt thereof) can be administered twice daily. For yet another example, Compound 1 (or the salt thereof) and ritonavir (or another CYP3A4 inhibitor, e.g., cobicistat) are co-formulated in a single composition and administered concurrently (e.g., once daily). For yet another example, Compound 1 (or the salt thereof), co-formulated with ritonavir (or another CYP3A4 inhibitor, e.g., cobicistat), is administered once daily, and Compound 2 (or the salt thereof) is administered twice daily. As a non-limiting example, the patient being treated can be infected with HCV genotype 1, such as genotype la or lb. As another non-limiting example, the patient can be infected with HCV genotype 2 or 3. As yet another non-limiting example, the patient can be a HCV-treatment naïve patient, a HCV-treatment experienced patient, an interferon non-responder (e.g., a null responder), or not a candidate for interferon treatment. The present technology also features the same aspect of the invention as described immediately above, except that the treatment does not include administering either ribavirin or interferon.

The direct-acting antiviral regimen of the present invention generally constitutes a complete treatment regimen, i.e., no subsequent interferon-containing regimen is intended. Thus, a treatment or use described herein generally does not include any subsequent interferon-containing treatment.

Other features, objects, and advantages of the present invention are apparent in the detailed description that follows. It should be understood, however, that the detailed description, while indicating preferred embodiments of the invention, are given by way of illustration only, not limitation. Various changes and modifications within the scope of the invention will become apparent to those skilled in the art from the detailed description.

### BRIEF DESCRIPTION OF THE DRAWINGS

FIG. 1 is a plot showing relative miR-122 expression levels in serum from patients treated with a direct-acting antiviral regimen for 12 weeks. MiR-122 expression levels were determined at baseline (“BASE”), week 2 on-treatment (“W2”), week 10 on-treatment (“W10”), and 8 weeks after completing the 12 week regimen (“Post Week 8”). The patients were categorized as (1) non-responders to the direct-acting antiviral regimen (“NR”; diamonds); (2) patients experiencing a viral rebound/relapse after treatment with the direct-acting antiviral regimen (“Rebound”; squares); (3) patients who had previously been exposed to an interferon-containing treatment regimen and ultimately achieved SVR after treatment with the direct-acting antiviral regimen (“Experienced SVR”; triangles); and (4) patients who had not been treated previously and achieved SVR after treatment with the direct-acting antiviral regimen (“Naïve SVR”; circles).

FIG. 2 is a plot showing relative baseline miR-21 expression levels in serum from patients who were subsequently treated with a direct-acting antiviral regimen for 12 weeks. The patients were categorized as (1) non-responders to the direct-acting antiviral regimen (“NR”; diamonds); (2) patients experiencing a viral rebound/relapse after treatment with the direct-acting antiviral regimen (“Rebound”; squares); (3) patients who had previously been exposed to an interferon-containing treatment regimen and ultimately achieved SVR after treatment with the direct-acting antiviral regimen (“Experienced SVR”; triangles); and (4) patients who had not been treated previously and achieved SVR after treatment with the direct-acting antiviral regimen (“Naïve SVR”; circles).

FIG. 3 is a plot showing miR-122 expression levels in serum from patients who achieved SVR after treatment with a direct-acting antiviral agent in combination with peginterferon α-2a and ribavirin (“IFN/RBV”). The patients received 3 days of monotherapy with a direct-acting antiviral agent: Compound 2 (squares) or Compound 4 (diamonds). The three days of monotherapy was followed by treatment with the same DAA with IFN/RBV for 12 weeks. miR-122 expression levels are shown at Day 1 (baseline) and Day 3 (following DAA monotherapy treatment) and at Weeks 4 and 10 during combined DAA/IFN/RBV therapy.

FIG. 4 is a plot showing miR-122 expression levels in serum from patients who were treated with a direct-acting antiviral regimen comprising a non-nucleoside polymerase inhibitor. The patients received 3 days of monotherapy with the polymerase inhibitor (either Compound 2 or Compound 4), followed by the same direct-acting antiviral compound with peginterferon α-2a and ribavirin for 12 weeks. miR-122 expression levels are shown at Day 1 (baseline) and Day 3 (following monotherapy treatment) and at Weeks 4 and 10 during combined DAA/IFN/RBV therapy. The patients were categorized as (1) non-responders (“Not SVR”; diamonds); and (2) patients who achieved SVR after treatment (“SVR”; squares).

FIG. 5 is a plot showing relative miR-122 expression levels in serum from HCV genotype 2-infected patients treated with a direct-acting antiviral regimen for 12 weeks. MiR-122 expression levels were determined at baseline (“Baseline”), day 3 on-treatment (“Day 3”), week 2 on-treatment (“Week 2”), week 10 on-treatment (“Week 10”), and 8 weeks after completing the 12 week regimen (“PT Week 8”). The patients were categorized as (1) patients experiencing viral breakthrough while on-treatment (“Breakthrough”); (2) patients experiencing a viral rebound/relapse after treatment with the direct-acting antiviral regimen (“Relapse”); and (3) patients who achieved SVR after treatment with the direct-acting antiviral regimen (“SVR”).

FIG. 6 is a plot showing relative miR-122 expression levels in serum from HCV genotype 3-infected patients treated with a direct-acting antiviral regimen for 12 weeks. MiR-122 expression levels were determined at baseline (“Baseline”), day 3 on-treatment (“Day 3”), week 2 on-treatment (“Week 2”), week 10 on-treatment (“Week 10”), and 8 weeks after completing the 12 week regimen (“PT Week 8”). The patients were categorized as (1) patients experiencing viral breakthrough while on-treatment (“Breakthrough”); (2) patients experiencing a viral rebound/relapse after treatment with the direct-acting antiviral regimen (“Relapse”); and (3) patients who achieved SVR after treatment with the direct-acting antiviral regimen (“SVR”).

FIG. 7 is a plot showing miR-122 expression levels in serum from HCV genotype 2-infected and HCV genotype 3-infected patients who were treated with a direct-acting antiviral regimen for 12 weeks. MiR-122 expression levels were determined at baseline (“Baseline”), day 3 on-treatment (“Day 3”), week 2 on-treatment (“Week 2”), week 10 on-treatment (“Week 10”), and 8 weeks after completing the 12 week regimen (“PT Week 8”). The patients were categorized as (1) patients who received ribavirin as part of the treatment (“RBV”); and (2) patients who did not receive ribavirin as part of the treatment (“no RBV”).

### DETAILED DESCRIPTION OF THE INVENTION

The present methods can include administering therapeutic agent 1 to a subject. Therapeutic agent 1 is Compound 1 or a pharmaceutically acceptable salt thereof.

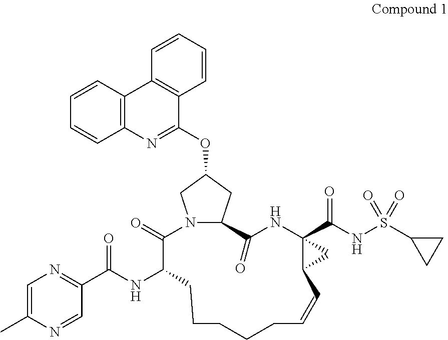 [SMILES: Cc1cnc(C(=O)N[C@H]2CCCCC/C=C\[C@@H]3C[C@@]3(C(=O)NS(=O)(=O)C3CC3)NC(=O)[C@@H]3C[C@@H](Oc4nc5ccccc5c5ccccc45)CN3C2=O)cn1]

Compound 1 is also known as (2R,6S,13aS,14aR,16aS,Z)-N-(cyclopropylsulfonyl)-6-(5-methylpyrazine-2-carboxamido)-5,16-dioxo-2-(phenanthridin-6-yloxy)-1,2,3,5,6,7,8,9,10,11,13a,14,14a,15,16,16a-hexadecahydrocyclopropa[e]pyrrolo[1,2-a][1,4]diazacyclopentadecine-14a-carboxamide. Compound 1 is a potent HCV protease inhibitor. The synthesis and formulation of Compound 1 are described in U.S. Patent Application Publication No. 2010/0144608, U.S. Provisional Application Ser. No. 61/339,964 filed on Mar. 10, 2010, and U.S. Patent Application Publication No. 2011/0312973 filed on Mar. 8, 2011. All of these applications are incorporated herein by reference in their entireties. Therapeutic agent 1 includes various salts of Compound 1. As non-limiting examples, therapeutic agent 1 may be administered in a total daily dosage amount of from 50 mg to 300 mg, preferably from 150 mg to 250 mg, and includes, but is not limited to, for example, 50 mg, 75 mg, 100 mg, 125 mg, 150 mg, 175 mg, 200 mg, 225 mg, 250 mg and suitable amounts there between. Preferably, therapeutic agent 1 is administered in a total daily dosage amount of 250 mg. The total daily dosage amount may be administered in one or more dosage forms and at one or more times daily. The total daily dosage amount may be administered once daily, twice daily, three times daily or at another frequency. For example, the total daily dosage amount may be divided between two dosage forms which are taken at different times during the day, thereby resulting in twice daily administration.

In preferred embodiments, ritonavir or another inhibitor of cytochrome P-450 (such as cobicistat) is administered in combination with therapeutic agent 1 to improve the pharmacokinetics of compound 1. Preferably the cytochrome 450 inhibitor is ritonavir administered at a daily dose of 50 mg to 400 mg, more preferably 100 mg. In some embodiments, therapeutic agent 1 is co-administered with the cytochrome 450 inhibitor. For instance, therapeutic agent 1 can be administered, for example and without limitation, concurrently with or sequentially with the cytochrome 450 inhibitor. Therapeutic agent 1 can be administered immediately before or after the administration of the cytochrome 450 inhibitor. A short delay or time gap between the administration of therapeutic agent 1 and the cytochrome 450 inhibitor is also contemplated.

Therapeutic agent 1 can be co-formulated with the cytochrome 450 inhibitor in a single dosage form. Non-limiting examples of suitable dosage forms include liquid or solid dosage forms. The dosage form may be a solid dosage form described in U.S. Publication No. 2011/0312973, entitled “Solid Compositions”, the entire content of which is incorporated herein by reference. For example, the dosage form may be a solid dosage form in which therapeutic agent 1 is molecularly dispersed in a matrix which comprises a pharmaceutically acceptable water-soluble polymer and a pharmaceutically acceptable surfactant. The cytochrome 450 inhibitor can also be molecularly dispersed in the matrix, or formulated in different form(s). As a non-limiting alternative, therapeutic agent 1 and the cytochrome 450 inhibitor can be formulated in two different dosage forms which can be provided as a combination to a subject.

The present methods can include administering therapeutic agent 2 to a subject. Therapeutic agent 2 is Compound 2 or a salt thereof.

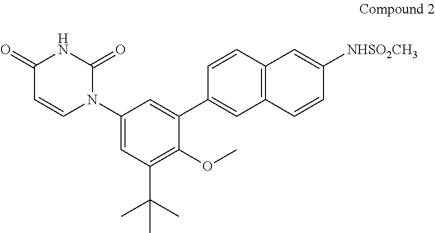

Compound 2 is also known N-(6-(3-tert-butyl-5-(2,4-dioxo-3,4-dihydropyrimidin-1(2H)-yl)-2-methoxyphenyl)naphthalen-2-yl)methanesulfonamide. As described in, for example, International Publication No. WO2009/039127, therapeutic agent 2 includes various salts of Compound 2, such as sodium salts, potassium salts, and choline salts. Therapeutic agent 2 also includes crystalline forms of Compound 2 and its salts such as solvate, hydrate, and solvent-free crystalline forms of Compound 2 and its salts. Compositions comprising therapeutic agent 2 can be prepared as described in, for example, International Publication No. WO2009/039127 which is incorporated by reference herein.

Therapeutic agent 2 may be administered as a free acid, salt or particular crystalline form of Compound 2. In some embodiments, therapeutic agent 2 is administered as a sodium salt. Therapeutic agent 2 may be administered in any suitable amount such as, for example, in doses of from 5 mg/kg to 30 mg/kg. As non-limiting examples, therapeutic agent 2 may be administered in a total daily dosage amount of from 300 mg to 1800 mg, or from 400 mg to 1600 mg, or from 600 mg to 1800 mg, or from 800 mg to 1600 mg or any amounts there between. In some embodiments, the total daily dosage amount for therapeutic agent 2 is 600 mg. In some embodiments, the total daily dosage amount for therapeutic agent 2 is 800 mg. In some embodiments, the total daily dosage amount for therapeutic agent 2 is 1200 mg. In some embodiments, the total daily dosage amount for therapeutic agent 2 is 1600 mg. For example, the total daily dosage amount may be divided between two dosage forms which are taken at different times during the day, thereby resulting in twice daily administration.

The present methods can include administering therapeutic agent 3 or a salt thereof to a subject. Therapeutic agent 3 is compound 3 or a salt thereof.

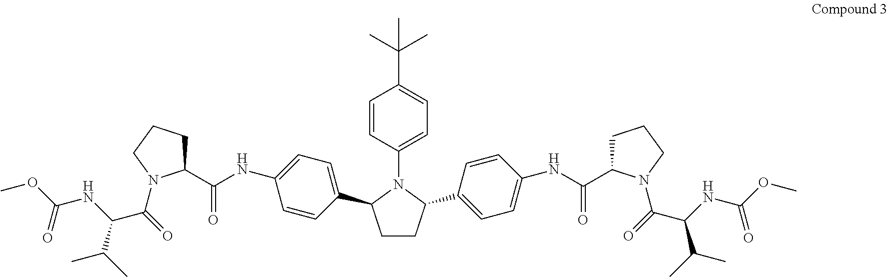

Compound 3 is also known as dimethyl (2S,2′S)-1,1′-((2S,2′S)-2,2′-(4,4′-(2S,5S)-1-(4-tert-butylphenyl)pyrrolidine-2,5, diyl)bis(4,1-phenylene))bis(azanediyl)bis(oxomethylene)bis(pyrrolidine-2,1-diyl)bis(3-methyl-1-oxobutane-2,1-diyl)dicarbamate. Compound 3 can be prepared as described in, for example, U.S. Publication No. 2010/0317568, which is incorporated herein by reference.

Therapeutic agent 3 may be administered as a free acid, or a salt form. Therapeutic agent 3 may be administered in any suitable amount such as, for example, in doses of from 0.1 mg/kg to 200 mg/kg body weight, or from 0.25 mg/kg to 100 mg/kg, or from 0.3 mg/kg to 30 mg/kg. As non-limiting examples, therapeutic agent 3 may be administered in a total daily dose amount of from 5 mg to 300 mg, or from 25 mg to 200 mg, or from 25 mg to 50 mg or any amounts there between. In some embodiments, the total daily dosage amount for therapeutic agent 3 is 25 mg.

The present methods can include administering therapeutic agent 4 to a subject. Therapeutic agent 4 is Compound 4 or a salt thereof.

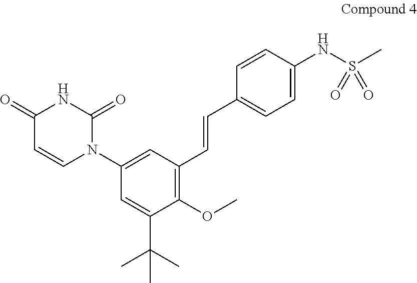 [SMILES: COc1c(/C=C/c2ccc(NS(C)(=O)=O)cc2)cc(-n2ccc(=O)[nH]c2=O)cc1C(C)(C)C]

Compound 4 is also known (E)-N-(4-(3-tert-butyl-5-(2,4-dioxo-3,4-dihydropyrimidin-1(2H)-yl)-2-methoxystyryl)phenyl)methanesulfonamide. Compound 4 is a polymerase inhibitor. As described in, for example, US Publication No. 2012/0189580, which is incorporated by reference herein, therapeutic agent 4 includes various salts of Compound 4, such as disodium salts, monopotassium salts, dipotassium salts, and monodiethylamine salts. Therapeutic agent 4 also includes crystalline forms of Compound 4 and its salts such as solvate, hydrate, and solvent-free crystalline forms of Compound 4 and its salts. Compositions comprising therapeutic agent 4 can be prepared as described in, for example, US Publication No. 2011/0237793, which is incorporated by reference herein.

Therapeutic agent 4 may be administered as a free acid, salt or particular crystalline form of Compound 4. In some embodiments, therapeutic agent 4 is administered as a disodium salt. In some embodiments, therapeutic agent 4 is administered as a monopotassium salt. In some embodiments, therapeutic agent 4 is administered as a dipotassium salt. In some embodiments, therapeutic agent 4 is administered as a monodiethylamine salt.

As non-limiting examples, therapeutic agent 4 may be administered in a total daily dosage amount of from 50 mg to 1200 mg or any amounts there between. As non-limiting examples, therapeutic agent 4 may be administered in a total daily dosage amount of from 100 mg to 600 mg or any amounts there between. In some embodiments, the total daily dosage amount for therapeutic agent 4 is 100 mg. In some embodiments, the total daily dosage amount for therapeutic agent 4 is 300 mg. In some embodiments, the total daily dosage amount for therapeutic agent 4 is 600 mg.

The total daily dosage amount may be administered in one or more dosage forms and at one or more times daily. The total daily dosage amount may be administered once daily, twice daily, three times daily or at another frequency. For example, the total daily dosage amount may be administered once daily or divided between two dosage forms which are taken at different times during the day, thereby resulting in twice daily administration.

The direct-acting antiviral regimen also can be co-administered with ribavirin, or a pro-drug thereof, in the same or separate pharmaceutical compositions. Ribavirin may include any suitable form or formulation of ribavirin. Exemplary formulations of ribavirin include COPEGUS®, REBETOL® and RIBASPHERE®. An exemplary pro-drug of ribavirin is taribavirin having the chemical name of 1-β-D-ribofuranosyl-1,2,4-triazole-3-carboxamidine. Ribavirin and taribavirin may be administered in accordance with ribavirin and taribavirin administration well known in the art. In some embodiments, COPEGUS® or REBETOL® is administered in a daily dosage amount of from 500 mg to 1500 mg in one dose or in divided doses. In some embodiments, COPEGUS® or REBETOL® is administered in a daily dosage amount of 800 mg. In some embodiments, REBETOL® is administered in a daily dosage amount of 1000 mg. In some embodiments, COPEGUS® or REBETOL® is administered in a daily dosage amount of 1200 mg. In some embodiments, REBETOL® is administered in a daily dosage amount of 1400 mg. Suitable dosages of ribavirin are dependent on the weight of the subject, for example 1000-1200 mg. Suitable total daily dosages of ribavirin include, but are not limited to 400 mg to 1400 mg a day, alternatively 800 mg to 1400 mg per day, alternatively 400 mg to 1200 mg, alternatively 800 mg to 1200 mg.

Current treatment of HCV includes a course of treatment of interferon, e.g. pegylated interferon (e.g., pegylated interferon-alpha-2a or pegylated interferon-alpha-2b, such as PEGASYS by Roche, or PEG-INTRON by Schering-Plough) and the antiviral drug ribavirin (e.g., COPEGUS by Roche, REBETOL by Schering-Plough, or RIBASPHERE by Three Rivers Pharmaceuticals). The treatment often lasts for 24-48 weeks, depending on hepatitis C virus genotype. Other interferons include, but are not limited to, interferon-alpha-2a (e.g., Roferon-A by Roche), interferon-alpha-2b (e.g., Intron-A by Schering-Plough), and interferon alfacon-1 (consensus interferon) (e.g., Infergen by Valeant). Less than 50% of patients with chronic HCV infection with genotype 1 virus respond to this therapy. Further, interferon therapy has many side effects that hinder patient compliance and results in premature discontinuation of the treatment.

The interferon/ribavirin-based treatment may be physically demanding, and can lead to temporary disability in some cases. A substantial proportion of patients will experience a panoply of side effects ranging from a “flu-like” syndrome (the most common, experienced for a few days after the weekly injection of interferon) to severe adverse events including anemia, cardiovascular events and psychiatric problems such as suicide or suicidal ideation. The latter are exacerbated by the general physiological stress experienced by the patients. Ribavirin also has a number of side effects, including, anemia, high pill burden (e.g. 5-6 pills a day split BID) and teratogenicity restricting use in women of childbearing age.

Various measures may be used to express the effectiveness of the present methods of HCV treatment. One such measure is rapid virological response (RVR), meaning that HCV is undetectable in the subject after 4 weeks of treatment, for example, after 4 weeks of administration of two or more of DAAs and ribavirin. Another measure is early virological response (EVR), meaning that the subject has >2 log₁₀ reduction in viral load after 12 weeks of treatment. Another measure is complete EVR (cEVR), meaning the HCV is undetectable in the serum of the subject after 12 weeks of treatment. Another measure is extended RVR (eRVR), meaning achievement of RVR and cEVR, that is, HCV is undetectable at week 4 and 12. Another measure is the presence or absence of detectable virus at the end of therapy (EOT). Another measure is (SVR), which, as used herein, means that the virus is undetectable at the end of therapy and for at least 8 weeks after the end of therapy (SVR8); preferably, the virus is undetectable at the end of therapy and for at least 12 weeks after the end of therapy (SVR12); more preferably, the virus is undetectable at the end of therapy and for at least 16 weeks after the end of therapy (SVR16); and highly preferably, the virus is undetectable at the end of therapy and for at least 24 weeks after the end of therapy (SVR24). SVR24 is often considered as a functional definition of cure; and a high rate of SVR at less than 24 week post-treatment (e.g., SVR8 or SVR12) can be predictive of a high rate of SVR24. Likewise, a high rate of SVR at less than 12 week post-treatment (e.g., SVR4 or SVR8) can be predictive of a high rate of SVR12. A high rate of undetectable virus at EOT (e.g., at week 8 or week 12) can also be indicative of a significant rate of SVR12 or SVR24.

In at least one aspect of the present invention, the effectiveness of the present methods of HCV treatment can be predicted using microRNA expression levels, such as miR-122 or miR-21 expression levels.

Thus, in certain aspects, the present invention provides methods of predicting an adequate clinical outcome of a patient infected with HCV, comprising detecting the expression level of miR-122 in a sample obtained from the patient. In some embodiments, an expression level of miR-122 in the sample that is equal to or less than a pre-determined control level predicts an adequate sustained response to treatment with a direct-acting antiviral regimen. In some embodiments, an expression level of miR-122 in the sample that is at least 1-fold less than a pre-determined control level predicts an adequate sustained response to treatment with a direct-acting antiviral regimen. In some embodiments, an expression level of miR-122 in the sample that is at least 2-fold less than a pre-determined control level predicts an adequate sustained response to treatment with a direct-acting antiviral regimen. In some embodiments, an expression level of miR-122 in the sample that is at least 3-fold less than a pre-determined control level predicts an adequate sustained response to treatment with a direct-acting antiviral regimen. In some embodiments, an expression level of miR-122 in the sample that is at least 4-fold less than a pre-determined control level predicts an adequate sustained response to treatment with a direct-acting antiviral regimen. In some embodiments, an expression level of miR-122 in the sample that is from 1-fold to 4-fold less than a pre-determined control level predicts an adequate sustained response to treatment with a direct-acting antiviral regimen. The pre-determined control level can be a range or a specific value. The pre-determined control level can be determined empirically, such as, by obtaining a mean expression level from a population of subjects. The population of subjects can be a population of healthy subjects or a population of HCV-infected subjects. The direct-acting antiviral regimen can comprise an HCV protease inhibitor, such as Compound 1, and an HCV polymerase inhibitor, such as Compound 2.

In some embodiments, an HCV-infected patient can be treated with a direct-acting antiviral regimen. Prior to the initiation of treatment with the direct-acting antiviral regimen, a sample is obtained from the patient to establish a baseline miR-122 expression level. In certain embodiments, the patient is treated with the direct-acting antiviral regimen when the baseline miR-122 expression level is equal to or less than a pre-determined control level. In certain other embodiments, the patient is treated with the direct-acting antiviral regimen when the baseline miR-122 expression level is at least 1-fold less than a pre-determined control level. In certain other embodiments, the patient is treated with the direct-acting antiviral regimen when the baseline miR-122 expression level is at least 2-fold less than a pre-determined control level. In certain other embodiments, the patient is treated with the direct-acting antiviral regimen when the baseline miR-122 expression level is at least 3-fold less than a pre-determined control level. In certain other embodiments, the patient is treated with the direct-acting antiviral regimen when the baseline miR-122 expression level is at least 4-fold less than a pre-determined control level. In certain other embodiments, the patient is treated with the direct-acting antiviral regimen when the baseline miR-122 expression level is from 1-fold to 4-fold less than a pre-determined control level. The pre-determined control level can be a range or a specific value. The pre-determined control level can be determined empirically, such as, by obtaining a mean expression level from a population of subjects. The population of subjects can be a population of healthy subjects or a population of HCV-infected subjects. The direct-acting antiviral regimen can comprise an HCV protease inhibitor and an HCV polymerase inhibitor. Preferably, the HCV protease inhibitor is Compound 1 and the HCV polymerase inhibitor is Compound 2.

In certain aspects, the present invention provides methods of predicting an adequate clinical outcome of a patient infected with HCV, comprising detecting the expression level of miR-21 in a sample obtained from the patient. In some embodiments, an expression level of miR-21 in the sample that is equal to or less than a pre-determined control level predicts an adequate sustained response to treatment with a direct-acting antiviral regimen. In some embodiments, an expression level of miR-21 in the sample that is at least 1-fold less than a pre-determined control level predicts an adequate sustained response to treatment with a direct-acting antiviral regimen. In some embodiments, an expression level of miR-21 in the sample that is at least 2-fold less than a pre-determined control level predicts an adequate sustained response to treatment with a direct-acting antiviral regimen. In some embodiments, an expression level of miR-21 in the sample that is at least 3-fold less than a pre-determined control level predicts an adequate sustained response to treatment with a direct-acting antiviral regimen. In some embodiments, an expression level of miR-21 in the sample that is at least 4-fold less than a pre-determined control level predicts an adequate sustained response to treatment with a direct-acting antiviral regimen. In some embodiments, an expression level of miR-21 in the sample that is from 1-fold to 4-fold less than a pre-determined control level predicts an adequate sustained response to treatment with a direct-acting antiviral regimen. The pre-determined control level can be a range or a specific value. The pre-determined control level can be determined empirically, such as, by obtaining a mean expression level from a population of subjects. The population of subjects can be a population of healthy subjects or a population of HCV-infected subjects. The direct-acting antiviral regimen can comprise an HCV protease inhibitor, such as Compound 1, and an HCV polymerase inhibitor, such as Compound 2.

In some embodiments, an HCV-infected patient can be treated with a direct-acting antiviral regimen. Prior to the initiation of treatment with the direct-acting antiviral regimen, a sample is obtained from the patient to establish a baseline miR-21 expression level. In certain embodiments, the patient is treated with the direct-acting antiviral regimen when the baseline miR-21 expression level is equal to or less than a pre-determined control level. In certain other embodiments, the patient is treated with the direct-acting antiviral regimen when the baseline miR-21 expression level is at least 1-fold less than a pre-determined control level. In certain other embodiments, the patient is treated with the direct-acting antiviral regimen when the baseline miR-21 expression level is at least 2-fold less than a pre-determined control level. In certain other embodiments, the patient is treated with the direct-acting antiviral regimen when the baseline miR-21 expression level is at least 3-fold less than a pre-determined control level. In certain other embodiments, the patient is treated with the direct-acting antiviral regimen when the baseline miR-21 expression level is at least 4-fold less than a pre-determined control level. In certain other embodiments, the patient is treated with the direct-acting antiviral regimen when the baseline miR-21 expression level is from 1-fold to 4-fold less than a pre-determined control level. The pre-determined control level can be a range or a specific value. The pre-determined control level can be determined empirically, such as, by obtaining a mean expression level from a population of subjects. The population of subjects can be a population of healthy subjects or a population of HCV-infected subjects. The direct-acting antiviral regimen can comprise an HCV protease inhibitor and an HCV polymerase inhibitor. Preferably, the HCV protease inhibitor is Compound 1 and the HCV polymerase inhibitor is Compound 2.

In certain other aspects, the present invention provides methods of predicting an inadequate clinical outcome of a patient infected with HCV, comprising detecting the expression level of miR-122 in a sample obtained from the patient. In some embodiments, an expression level of miR-122 in the sample that is greater than a pre-determined control level predicts an inadequate sustained response to treatment with a direct-acting antiviral regimen. In some embodiments, an expression level of miR-122 in the sample that is at least 1-fold greater than a pre-determined control level predicts an inadequate sustained response to treatment with a direct-acting antiviral regimen. In some embodiments, an expression level of miR-122 in the sample that is at least 2-fold greater than a pre-determined control level predicts an inadequate sustained response to treatment with a direct-acting antiviral regimen. In some embodiments, an expression level of miR-122 in the sample that is at least 3-fold greater than a pre-determined control level predicts an inadequate sustained response to treatment with a direct-acting antiviral regimen. In some embodiments, an expression level of miR-122 in the sample that is at least 4-fold greater than a pre-determined control level predicts an inadequate sustained response to treatment with a direct-acting antiviral regimen. In some embodiments, an expression level of miR-122 in the sample that is from 1-fold to 4-fold greater than a pre-determined control level predicts an inadequate sustained response to treatment with a direct-acting antiviral regimen. The pre-determined control level can be a range or a specific value. The pre-determined control level can be determined empirically, such as, by obtaining a mean expression level from a population of subjects. The population of subjects can be a population of healthy subjects or a population of HCV-infected subjects. The direct-acting antiviral regimen can comprise an HCV protease inhibitor, such as Compound 1, and an HCV polymerase inhibitor, such as Compound 2.

In still other aspects, the present invention provides methods of predicting an inadequate clinical outcome of a patient infected with HCV, comprising detecting the expression level of miR-21 in a sample obtained from the patient. In some embodiments, an expression level of miR-21 in the sample that is greater than a pre-determined control level predicts an inadequate sustained response to treatment with a direct-acting antiviral regimen. In some embodiments, an expression level of miR-21 in the sample that is at least 1-fold greater than a pre-determined control level predicts an inadequate sustained response to treatment with a direct-acting antiviral regimen. In some embodiments, an expression level of miR-21 in the sample that is at least 2-fold greater than a pre-determined control level predicts an inadequate sustained response to treatment with a direct-acting antiviral regimen. In some embodiments, an expression level of miR-21 in the sample that is at least 3-fold greater than a pre-determined control level predicts an inadequate sustained response to treatment with a direct-acting antiviral regimen. In some embodiments, an expression level of miR-21 in the sample that is at least 4-fold greater than a pre-determined control level predicts an inadequate sustained response to treatment with a direct-acting antiviral regimen. In some embodiments, an expression level of miR-21 in the sample that is from 1-fold to 4-fold greater than a pre-determined control level predicts an inadequate sustained response to treatment with a direct-acting antiviral regimen. The pre-determined control level can be a range or a specific value. The pre-determined control level can be determined empirically, such as, by obtaining a mean expression level from a population of subjects. The population of subjects can be a population of healthy subjects or a population of HCV-infected subjects. The direct-acting antiviral regimen can comprise an HCV protease inhibitor, such as Compound 1, and an HCV polymerase inhibitor, such as Compound 2.

In some embodiments, an HCV-infected patient can be treated with a direct-acting antiviral regimen for 12 or more weeks. For example, the direct-acting antiviral regimen can be administered from 13 to 24 weeks, and, more particularly, for 12, 13, 14, 15, 16, 17, 18, 19, 20, 21, 22, 23 weeks, or 24 weeks. Prior to the initiation of treatment with the direct-acting antiviral regimen, a sample is obtained from the patient to establish a baseline miR-122 expression level. In certain embodiments, the patient is treated with the direct-acting antiviral regimen for 12 or more weeks when the baseline miR-122 expression level is greater than a pre-determined control level. In certain other embodiments, the patient is treated with the direct-acting antiviral regimen for 12 or more weeks when the baseline miR-122 expression level is at least 1-fold greater than a pre-determined control level. In certain other embodiments, the patient is treated with the direct-acting antiviral regimen for 12 or more weeks when the baseline miR-122 expression level is at least 2-fold greater than a pre-determined control level. In certain other embodiments, the patient is treated with the direct-acting antiviral regimen for 12 or more weeks when the baseline miR-122 expression level is at least 3-fold greater than a pre-determined control level. In certain other embodiments, the patient is treated with the direct-acting antiviral regimen for 12 or more weeks when the baseline miR-122 expression level is at least 4-fold greater than a pre-determined control level. In certain other embodiments, the patient is treated with the direct-acting antiviral regimen for 12 or more weeks when the baseline miR-122 expression level is from 1-fold to 4-fold greater than a pre-determined control level. The pre-determined control level can be a range or a specific value. The pre-determined control level can be determined empirically, such as, by obtaining a mean expression level from a population of subjects. The population of subjects can be a population of healthy subjects or a population of HCV-infected subjects. The direct-acting antiviral regimen can comprise an HCV protease inhibitor and an HCV polymerase inhibitor. Preferably, the HCV protease inhibitor is Compound 1 and the HCV polymerase inhibitor is Compound 2. Preferably, the direct-acting antiviral regimen, and, optionally, ribavirin are administered in effective amounts and for an effective duration to provide a desired measure of effectiveness in the patient. Preferably, the direct-acting antiviral regimen is administered for a duration effective to provide a desired measure of effectiveness in a patient having a high baseline miR-122 expression level (e.g., prior to initiation of the direct-acting antiviral regimen).

In some embodiments, an HCV-infected patient can be treated with a direct-acting antiviral regimen for 12 or more weeks. For example, the direct-acting antiviral regimen can be administered from 13 to 24 weeks, and, more particularly, for 12, 13, 14, 15, 16, 17, 18, 19, 20, 21, 22, 23 weeks, or 24 weeks. Prior to the initiation of treatment with the direct-acting antiviral regimen, a sample is obtained from the patient to establish a baseline miR-21 expression level. In certain embodiments, the patient is treated with the direct-acting antiviral regimen for 12 or more weeks when the baseline miR-21 expression level is greater than a pre-determined control level. In certain other embodiments, the patient is treated with the direct-acting antiviral regimen for 12 or more weeks when the baseline miR-21 expression level is at least 1-fold greater than a pre-determined control level. In certain other embodiments, the patient is treated with the direct-acting antiviral regimen for 12 or more weeks when the baseline miR-21 expression level is at least 2-fold greater than a pre-determined control level. In certain other embodiments, the patient is treated with the direct-acting antiviral regimen for 12 or more weeks when the baseline miR-21 expression level is at least 3-fold greater than a pre-determined control level. In certain other embodiments, the patient is treated with the direct-acting antiviral regimen for 12 or more weeks when the baseline miR-21 expression level is at least 4-fold greater than a pre-determined control level. In certain other embodiments, the patient is treated with the direct-acting antiviral regimen for 12 or more weeks when the baseline miR-21 expression level is from 1-fold to 4-fold greater than a pre-determined control level. The pre-determined control level can be a range or a specific value. The pre-determined control level can be determined empirically, such as, by obtaining a mean expression level from a population of subjects. The population of subjects can be a population of healthy subjects or a population of HCV-infected subjects. The direct-acting antiviral regimen can comprise an HCV protease inhibitor and an HCV polymerase inhibitor. Preferably, the HCV protease inhibitor is Compound 1 and the HCV polymerase inhibitor is Compound 2. Preferably, the direct-acting antiviral regimen, and, optionally, ribavirin are administered in effective amounts and for an effective duration to provide a desired measure of effectiveness in the patient. Preferably, the direct-acting antiviral regimen is administered for a duration effective to provide a desired measure of effectiveness in a patient having a high baseline miR-21 expression level (e.g., prior to initiation of the direct-acting antiviral regimen).

In some embodiments, the effectiveness of the present methods of HCV treatment can be assessed using microRNA expression levels, such as miR-122 or miR-21 expression levels. For example, assessment of a patient's on-treatment microRNA expression levels can enable a prognosis that the patient will achieve SVR. In some embodiments, a patient may not achieve SVR if that patient's on-treatment microRNA expression levels are greater than or equal to the patient's microRNA expression levels prior to the initiation of treatment. For example, a patient's miR-122 (or miR-21) expression level may be determined prior to the initiation of treatment with a direct-acting antiviral regimen (i.e., baseline) and after 2 to 10 weeks of treatment with the direct-acting antiviral regimen (i.e., on-treatment). A patient may not achieve an adequate response to the direct-acting antiviral regimen if that patient's on-treatment miR-122 (or miR-21) expression level is greater than or equal to the patient's baseline miR-122 (or miR-21) expression level. Thus, a second (different) direct-acting antiviral regimen and/or longer duration of therapy may be indicated where a patient's on-treatment miR-122 (or miR-21) expression level is greater than or equal to the patient's baseline miR-122 (or miR-21) expression level.

Thus, in certain aspects, the present invention provides methods of determining responsiveness to a direct-acting antiviral regimen, comprising detecting the on-treatment miR-122 expression level in a sample obtained from the patient. The direct-acting antiviral regimen can comprise an HCV protease inhibitor, such as Compound 1, and an HCV polymerase inhibitor, such as Compound 2. In certain embodiments, the direct-acting antiviral regimen can also comprise an HCV NS5A inhibitor, such as Compound 1, and an HCV polymerase inhibitor, such as Compound 3. The on-treatment expression level can be determined in a sample obtained from the patient after the initiation of treatment with the direct-acting antiviral regimen. For example, the on-treatment expression level can be determined in a sample obtained from 1 week to 11 weeks after the initiation of treatment with the direct-acting antiviral regimen and, more particularly, 1, 2, 3, 4, 5, 6, 7, 8, 9, 10, or 11 weeks after the initiation of treatment with the direct-acting antiviral regimen. In some embodiments, an on-treatment miR-122 expression level in the sample is compared to a pre-determined control level. The pre-determined control level can be a range or a specific value. The pre-determined control level can be determined empirically, such as, by obtaining a mean expression level from a population of subjects. The population of subjects can be a population of healthy subjects or a population of HCV-infected subjects. In other embodiments, the on-treatment miR-122 expression level in the sample is compared to a miR-122 expression level from a sample obtained prior to initiation of the direct-acting antiviral regimen (i.e., a baseline expression level).

In some embodiments, an on-treatment miR-122 expression level that is less than a pre-determined control level indicates that a patient is responsive to treatment with a direct-acting antiviral regimen. In other embodiments, an on-treatment miR-122 expression level in the sample that is less than the patient's baseline miR-122 expression level indicates that a patient is responsive to treatment with a direct-acting antiviral regimen. In certain embodiments, treatment of a patient with the direct-acting antiviral regimen is continued when the on-treatment miR-122 expression level is less than a pre-determined control level. In certain other embodiments, treatment of a patient with the direct-acting antiviral regimen is continued when the on-treatment miR-122 expression level is less than the patient's baseline miR-122 expression level.

In some embodiments, an on-treatment miR-122 expression level that is greater than a pre-determined control level indicates that a patient is non-responsive to treatment with a direct-acting antiviral regimen. In other embodiments, an on-treatment miR-122 expression level in the sample that is greater than the patient's baseline miR-122 expression level indicates that a patient is non-responsive to treatment with a direct-acting antiviral regimen. In certain embodiments, treatment of a patient with the direct-acting antiviral regimen is discontinued when the on-treatment miR-122 expression level is greater than a pre-determined control level. In certain other embodiments, treatment of a patient with the direct-acting antiviral regimen is discontinued when the on-treatment miR-122 expression level is greater than the patient's baseline miR-122 expression level.

In certain embodiments, the dose of one or more of the agents included in the direct-acting antiviral regimen is increased when the on-treatment miR-122 expression level is greater than a pre-determined control level. In certain embodiments, one or more direct acting antiviral agents is added to the direct-acting antiviral regimen when the on-treatment miR-122 expression level is greater than a pre-determined control level. In certain embodiments, the dose of one or more of the agents included in the direct-acting antiviral regimen is increased when the on-treatment miR-122 expression level is greater than the patient's baseline miR-122 expression level. In certain embodiments, one or more direct acting antiviral agents is added to the direct-acting antiviral regimen when the on-treatment miR-122 expression level is greater than the patient's baseline miR-122 expression level.

In certain other aspects, the present invention provides methods of determining responsiveness by a patient to a direct-acting antiviral regimen, comprising detecting the on-treatment miR-21 expression level in a sample obtained from the patient. The direct-acting antiviral regimen can comprise an HCV protease inhibitor, such as Compound 1, and an HCV polymerase inhibitor, such as Compound 2. In certain embodiments, the direct-acting antiviral regimen can also comprise an HCV NS5A inhibitor, such as Compound 1, and an HCV polymerase inhibitor, such as Compound 3. The on-treatment expression level can be determined in a sample obtained from the patient after the initiation of treatment with the direct-acting antiviral regimen. For example, the on-treatment expression level can be determined in a sample obtained from 1 week to 11 weeks after the initiation of treatment with the direct-acting antiviral regimen and, more particularly, 1, 2, 3, 4, 5, 6, 7, 8, 9, 10, or 11 weeks after the initiation of treatment with the direct-acting antiviral regimen. In some embodiments, an on-treatment miR-21 expression level in the sample is compared to a pre-determined control level. The pre-determined control level can be a range or a specific value. The pre-determined control level can be determined empirically, such as, by obtaining a mean expression level from a population of subjects. The population of subjects can be a population of healthy subjects or a population of HCV-infected subjects. In other embodiments, the on-treatment miR-21 expression level in the sample is compared to a miR-21 expression level from a sample obtained prior to initiation of the direct-acting antiviral regimen (i.e., a baseline expression level).

In some embodiments, an on-treatment miR-21 expression level that is less than a pre-determined control level indicates that a patient is responsive to treatment with a direct-acting antiviral regimen. In other embodiments, an on-treatment miR-21 expression level in the sample that is less than the patient's baseline miR-21 expression level indicates that a patient is responsive to treatment with a direct-acting antiviral regimen. In certain embodiments, treatment of a patient with the direct-acting antiviral regimen is continued when the on-treatment miR-21 expression level is less than a pre-determined control level. In certain other embodiments, treatment of a patient with the direct-acting antiviral regimen is continued when the on-treatment miR-21 expression level is less than the patient's baseline miR-21 expression level.

In some embodiments, an on-treatment miR-21 expression level that is greater than a pre-determined control level indicates that a patient is non-responsive to treatment with a direct-acting antiviral regimen. In other embodiments, an on-treatment miR-21 expression level in the sample that is greater than the patient's baseline miR-21 expression level indicates that a patient is non-responsive to treatment with a direct-acting antiviral regimen. In certain embodiments, treatment of a patient with the direct-acting antiviral regimen is discontinued when the on-treatment miR-21 expression level is greater than a pre-determined control level. In certain other embodiments, treatment of a patient with the direct-acting antiviral regimen is discontinued when the on-treatment miR-21 expression level is greater than the patient's baseline miR-21 expression level.

In certain embodiments, the dose of one or more of the agents included in the direct-acting antiviral regimen is increased when the on-treatment miR-21 expression level is greater than a pre-determined control level. In certain embodiments, one or more direct acting antiviral agents is added to the direct-acting antiviral regimen when the on-treatment miR-21 expression level is greater than a pre-determined control level. In certain embodiments, the dose of one or more of the agents included in the direct-acting antiviral regimen is increased when the on-treatment miR-21 expression level is greater than the patient's baseline miR-21 expression level. In certain embodiments, one or more direct acting antiviral agents is added to the direct-acting antiviral regimen when the on-treatment miR-21 expression level is greater than the patient's baseline miR-21 expression level.

In at least one aspect of the present invention, the effectiveness of a polymerase inhibitor-containing regimen can be assessed using microRNA expression levels, such as miR-122 expression levels.

In some embodiments, the effectiveness of a polymerase inhibitor-containing regimen can be assessed using miR-122 expression levels. For example, assessment of a patient's on-treatment miR-122 expression levels can enable a prognosis that the patient will achieve SVR. In some embodiments, a patient may not achieve SVR if that patient's on-treatment miR-122 expression levels are not less than the patient's miR-122 expression levels prior to the initiation of treatment. For example, a patient's miR-122 expression level may be determined prior to the initiation of treatment with a polymerase inhibitor-containing regimen (i.e., baseline) and after 3 days to 11 weeks of treatment with the polymerase inhibitor-containing regimen (i.e., on-treatment). More particularly, a patient's miR-122 expression level may be determined after 3, 4, 5, or 6 days or 1, 2, 3, 4, 5, 6, 7, 8, 9, 10, or 11 weeks of treatment with the polymerase inhibitor-containing regimen. A patient may not achieve an adequate response to the polymerase inhibitor-containing regimen if that patient's on-treatment miR-122 expression level is not less than the patient's baseline miR-122 expression level. Thus, a second (different) direct-acting antiviral regimen and/or longer duration of therapy may be indicated where a patient's on-treatment miR-122 expression level is not less than the patient's baseline miR-122 expression level.

Thus, in certain aspects, the present invention provides methods of determining responsiveness to a polymerase inhibitor-containing regimen, comprising detecting the on-treatment miR-122 expression level in a sample obtained from the patient. The direct-acting antiviral regimen can comprise an HCV protease inhibitor, such as Compound 1, and an HCV polymerase inhibitor, such as Compound 2 or Compound 4. The on-treatment expression level can be determined in a sample obtained from the patient after the initiation of treatment with the polymerase inhibitor-containing regimen. For example, the on-treatment expression level can be determined in a sample obtained from 3 days to 11 weeks after the initiation of treatment with the direct-acting antiviral regimen and, more particularly, 3, 4, 5, or 6 days or 1, 2, 3, 4, 5, 6, 7, 8, 9, 10, or 11 weeks after the initiation of treatment with the direct-acting antiviral regimen. In some embodiments, an on-treatment miR-122 expression level in the sample is compared to a pre-determined control level. The pre-determined control level can be a range or a specific value. The pre-determined control level can be determined empirically, such as, by obtaining a mean expression level from a population of subjects. The population of subjects can be a population of healthy subjects or a population of HCV-infected subjects. In other embodiments, the on-treatment miR-122 expression level in the sample is compared to a miR-122 expression level from a sample obtained prior to initiation of the polymerase inhibitor-containing regimen (i.e., a baseline expression level).

In some embodiments, an on-treatment miR-122 expression level that is less than a pre-determined control level indicates that a patient is responsive to treatment with a polymerase inhibitor-containing regimen. In other embodiments, an on-treatment miR-122 expression level in the sample that is less than the patient's baseline miR-122 expression level indicates that a patient is responsive to treatment with a polymerase inhibitor-containing regimen. In certain embodiments, treatment of a patient with the polymerase inhibitor-containing regimen is continued when the on-treatment miR-122 expression level is less than a pre-determined control level. In certain embodiments, treatment of a patient with the polymerase inhibitor-containing regimen is stopped or the dose of the polymerase inhibitor is increased or a different polymerase inhibitor is used if the on-treatment miR-122 expression level is greater than or substantially equal to a pre-determined control level. In certain other embodiments, treatment of a patient with the polymerase inhibitor-containing regimen is continued when the on-treatment miR-122 expression level is less than the patient's baseline miR-122 expression level. In certain embodiments, treatment of a patient with the polymerase inhibitor-containing regimen is stopped or the dose of the polymerase inhibitor is increased or a different polymerase inhibitor is used if the on-treatment miR-122 expression level is greater than or substantially equal to the patient's baseline miR-122 expression level.

It was unexpectedly discovered that patients with an inadequate sustained response (e.g., relapse) following treatment with a direct-acting antiviral regimen had higher baseline miR-122 and miR-21 expression levels as compared to patients that achieved an adequate sustained response (e.g., SVR) following treatment with a direct-acting antiviral regimen. Thus, baseline miR-122 and/or miR-21 levels can be used as a biomarker for individuals that may relapse or otherwise inadequately respond to treatment with a direct-acting antiviral regimen. Such information can be used to guide therapy.

In some embodiments, an HCV-infected patient presenting with high baseline miR-122 and/or miR-21 expression levels can be treated with a direct-acting antiviral regimen designed to effectively treat such patients. In some instances, the direct-acting antiviral regimen may be administered for 12 or more weeks. For example, the direct-acting antiviral regimen can be administered from 13 to 24 weeks, and, more particularly, for 12, 13, 14, 15, 16, 17, 18, 19, 20, 21, 22, 23 weeks, or 24 weeks. In other instances, the direct-acting antiviral regimen may comprise at least two DAAs. Each DAA can be selected from, for example, HCV protease inhibitors, HCV polymerase inhibitors, or HCV NS5A inhibitors.

It also was surprising that serum miR-122 levels changed in response to HCV therapy. More particularly, serum miR-122 levels decrease in response to treatment with a direct-acting antiviral regimen. However, serum miR-122 levels returned to baseline in subjects who relapsed or failed treatment. These results suggest that serum levels of miR-122 were reflective of HCV levels. Thus, on-treatment miR-122 and/or miR-21 levels can be used as a biomarker for individuals that are not responsive to treatment with a direct-acting antiviral regimen. Such information can be used to guide therapy.

In some embodiments, treatment with a direct-acting antiviral regimen may be discontinued in a patient whose serum miR-122 levels returned to baseline while on treatment. Alternatively, a patient whose serum miR-122 levels returned to baseline while on treatment with a first direct-acting antiviral regimen can be treated with a second direct-acting antiviral regimen designed to effectively treat such patients. For example, the second direct-acting antiviral regimen may comprise at least two DAAs. Each DAA can be selected from, for example, HCV protease inhibitors, HCV polymerase inhibitors, or HCV NS5A inhibitors. Alternatively, a patient whose serum miR-122 levels returned to baseline while on treatment with a first direct-acting antiviral regimen can be treated with a higher dose of at least one of the DAAs of the first direct-acting antiviral regimen. As further discussed herein, in certain embodiments, the patient is infected with HCV genotype 1, HCV genotype 2, or HCV genotype 3.

Certain aspects of the present invention comprise treatment with a direct-acting antiviral regimen. The direct-acting antiviral regimen can comprise, for example, one or more DAAs. For instance, the direct-acting antiviral regimen can comprise a combination of two or more DAAs. The combination of two or more DAAs can be a combination of at least one HCV protease inhibitor and at least one HCV polymerase inhibitor (e.g., a combination of at least one HCV protease inhibitor and at least one non-nucleoside polymerase inhibitor, or a combination of at least one HCV protease inhibitor and at least one nucleoside or nucleotide polymerase inhibitor, or a combination of at least one HCV protease inhibitor, at least one nucleoside or nucleotide polymerase inhibitor and at least one non-nucleoside inhibitor). For another instance, the combination of two or more DAAs can be a combination of at least one HCV protease inhibitor and at least one HCV NS5A inhibitor. For still another instance, the combination of two or more DAAs can be a combination of at least one HCV protease inhibitor, at least one HCV polymerase inhibitor, and at least one HCV NS5A inhibitor. For another instance, the combination of two or more DAAs can be a combination of at least two HCV polymerase inhibitors (e.g., a combination of at least two nucleoside polymerase inhibitors, or a combination of at least one nucleoside or nucleotide polymerase inhibitor and at least one non-nucleoside or nucleotide polymerase inhibitor, or a combination of at least two non-nucleoside polymerase inhibitors). For another instance, the combination of two or more DAAs can be a combination of at least two HCV protease inhibitors. For another instance, the combination of two or more DAAs can be a combination of at least two HCV NS5A inhibitors. For another instance, the combination of two or more DAAs can be a combination of at least one HCV polymerase inhibitor and at least one NS5A inhibitor (e.g., a combination of at least one HCV NS5A inhibitor and at least one non-nucleoside or nucleotide polymerase inhibitor, or a combination of at least one HCV NS5A inhibitor and at least one nucleoside or nucleotide polymerase inhibitor, or a combination of at least one HCV NS5A inhibitor, at least one nucleoside or nucleotide polymerase inhibitor and at least one non-nucleoside polymerase inhibitor).

In one embodiment, the present invention features methods of treating HCV comprising administering to a patient in need thereof a direct-acting antiviral regimen, wherein the patient is tested for miR-122 level in blood prior to the treatment. The direct-acting antiviral regimen comprises a combination of Compound 1 (or a salt thereof) and Compound 2 (or a salt thereof). Compound 1 (or a salt thereof) can be co-administered or co-formulated with ritonavir. Preferably, the treatment lasts 12 weeks, and the patient's pre-treatment miR-122 level in blood is significantly lower than a mean pre-treatment miR-122 blood level in HCV patients who rebound after the 12-week treatment. Also preferably, the treatment lasts 12 weeks, and the patient's pre-treatment miR-122 level in blood is at least 1-fold lower than a mean pre-treatment miR-122 blood level in HCV patients who rebound after the 12-week treatment. Also preferably, the treatment lasts 12 weeks, and the patient's pre-treatment miR-122 level in blood is at least 2-fold lower than a mean pre-treatment miR-122 blood level in HCV patients who rebound after the 12-week treatment. Also preferably, the treatment lasts 12 weeks, and the patient's pre-treatment miR-122 level in blood is at least 3-fold lower than a mean pre-treatment miR-122 blood level in HCV patients who rebound after the 12-week treatment. Also preferably, the treatment lasts 12 weeks, and the patient's pre-treatment miR-122 level in blood is at least 4-fold lower than a mean pre-treatment miR-122 blood level in HCV patients who rebound after the 12-week treatment. Also preferably, the treatment lasts 12 weeks, and the patient's pre-treatment miR-122 level in blood is from 1-fold to 4-fold lower than a mean pre-treatment miR-122 blood level in HCV patients who rebound after the 12-week treatment. For example, the direct-acting antiviral regimen can comprise Compound 1 (or a salt thereof) dosed at 150 mg QD, Compound 2 (or a salt thereof) dosed at 400 mg BID, ritonavir dosed at 100 mg QD, and, optionally, ribavirin dosed once or twice daily. The regimen can further comprise Compound 3 (or a salt thereof). As non-limiting examples, Compound 3 (or a salt thereof) can be administered in a total daily dose amount of from 5 mg to 300 mg, or from 25 mg to 200 mg, or from 25 mg to 50 mg or any amounts there between. In some embodiments, the total daily dosage amount for Compound 3 (or a salt thereof) is 25 mg. The present technology also features the same aspect of the invention as described immediately above, except that pre-treatment miR-21 level in blood is used in lieu of pre-treatment miR-122 level in blood.

In another embodiment, the present invention features methods of treating HCV comprising administering to a patient in need thereof a direct-acting antiviral regimen, wherein the patient is tested for miR-122 level in blood prior to the treatment. The direct-acting antiviral regimen comprises GS-7977. Preferably, the treatment lasts 12 weeks, and the patient's pre-treatment miR-122 level in blood is significantly lower than a mean pre-treatment miR-122 blood level in HCV patients who rebound after the 12-week treatment. Also preferably, the treatment lasts 12 weeks, and the patient's pre-treatment miR-122 level in blood is at least 1-fold lower than a mean pre-treatment miR-122 blood level in HCV patients who rebound after the 12-week treatment. Also preferably, the treatment lasts 12 weeks, and the patient's pre-treatment miR-122 level in blood is at least 2-fold lower than a mean pre-treatment miR-122 blood level in HCV patients who rebound after the 12-week treatment. Also preferably, the treatment lasts 12 weeks, and the patient's pre-treatment miR-122 level in blood is at least 3-fold lower than a mean pre-treatment miR-122 blood level in HCV patients who rebound after the 12-week treatment. Also preferably, the treatment lasts 12 weeks, and the patient's pre-treatment miR-122 level in blood is at least 4-fold lower than a mean pre-treatment miR-122 blood level in HCV patients who rebound after the 12-week treatment. Also preferably, the treatment lasts 12 weeks, and the patient's pre-treatment miR-122 level in blood is from 1-fold to 4-fold lower than a mean pre-treatment miR-122 blood level in HCV patients who rebound after the 12-week treatment. For example, the direct-acting antiviral regimen can comprise GS-7977 dosed at 400 mg QD, and, optionally, ribavirin dosed once or twice daily. The regimen can be administered for a duration of 8 to 24 weeks, including for example, 12 or 16 weeks. The present technology also features the same aspect of the invention as described immediately above, except that pre-treatment miR-21 level in blood is used in lieu of pre-treatment miR-122 level in blood.

In another embodiment, the present invention features methods of treating HCV comprising administering to a patient in need thereof a direct-acting antiviral regimen, wherein the patient is tested for miR-122 level in blood prior to the treatment. The direct-acting antiviral regimen comprises a combination of GS-7977 and GS-5885. Preferably, the treatment lasts 12 weeks, and the patient's pre-treatment miR-122 level in blood is significantly lower than a mean pre-treatment miR-122 blood level in HCV patients who rebound after the 12-week treatment. Also preferably, the treatment lasts 12 weeks, and the patient's pre-treatment miR-122 level in blood is at least 1-fold lower than a mean pre-treatment miR-122 blood level in HCV patients who rebound after the 12-week treatment. Also preferably, the treatment lasts 12 weeks, and the patient's pre-treatment miR-122 level in blood is at least 2-fold lower than a mean pre-treatment miR-122 blood level in HCV patients who rebound after the 12-week treatment. Also preferably, the treatment lasts 12 weeks, and the patient's pre-treatment miR-122 level in blood is at least 3-fold lower than a mean pre-treatment miR-122 blood level in HCV patients who rebound after the 12-week treatment. Also preferably, the treatment lasts 12 weeks, and the patient's pre-treatment miR-122 level in blood is at least 4-fold lower than a mean pre-treatment miR-122 blood level in HCV patients who rebound after the 12-week treatment. Also preferably, the treatment lasts 12 weeks, and the patient's pre-treatment miR-122 level in blood is from 1-fold to 4-fold lower than a mean pre-treatment miR-122 blood level in HCV patients who rebound after the 12-week treatment. For example, the direct-acting antiviral regimen can comprise GS-7977 dosed at 400 mg QD, GS-5885 dosed at 90 mg QD, and, optionally, ribavirin dosed once or twice daily. The regimen can be administered for a duration of 6 to 24 weeks, including for example, 6, 8, or 12 weeks. The present technology also features the same aspect of the invention as described immediately above, except that pre-treatment miR-21 level in blood is used in lieu of pre-treatment miR-122 level in blood.

In another embodiment, the present invention features methods of treating HCV comprising administering to a patient in need thereof a direct-acting antiviral regimen, wherein the patient is tested for miR-122 level in blood prior to the treatment. The direct-acting antiviral regimen comprises a combination of GS-7977 and GS-9669. Preferably, the treatment lasts 12 weeks, and the patient's pre-treatment miR-122 level in blood is significantly lower than a mean pre-treatment miR-122 blood level in HCV patients who rebound after the 12-week treatment. Also preferably, the treatment lasts 12 weeks, and the patient's pre-treatment miR-122 level in blood is at least 1-fold lower than a mean pre-treatment miR-122 blood level in HCV patients who rebound after the 12-week treatment. Also preferably, the treatment lasts 12 weeks, and the patient's pre-treatment miR-122 level in blood is at least 2-fold lower than a mean pre-treatment miR-122 blood level in HCV patients who rebound after the 12-week treatment. Also preferably, the treatment lasts 12 weeks, and the patient's pre-treatment miR-122 level in blood is at least 3-fold lower than a mean pre-treatment miR-122 blood level in HCV patients who rebound after the 12-week treatment. Also preferably, the treatment lasts 12 weeks, and the patient's pre-treatment miR-122 level in blood is at least 4-fold lower than a mean pre-treatment miR-122 blood level in HCV patients who rebound after the 12-week treatment. Also preferably, the treatment lasts 12 weeks, and the patient's pre-treatment miR-122 level in blood is from 1-fold to 4-fold lower than a mean pre-treatment miR-122 blood level in HCV patients who rebound after the 12-week treatment. For example, the direct-acting antiviral regimen can comprise GS-7977 dosed at 400 mg QD, GS-9669 dosed at 500 mg QD, and, optionally, ribavirin dosed once or twice daily. The regimen can be administered for a duration of 8 to 24 weeks, including for example, 12 weeks. The present technology also features the same aspect of the invention as described immediately above, except that pre-treatment miR-21 level in blood is used in lieu of pre-treatment miR-122 level in blood.

In another embodiment, the present invention features methods of treating HCV comprising administering to a patient in need thereof a direct-acting antiviral regimen, wherein the patient is tested for miR-122 level in blood prior to the treatment. The direct-acting antiviral regimen comprises a combination of GS-7977 and daclatasvir. Preferably, the treatment lasts 12 weeks, and the patient's pre-treatment miR-122 level in blood is significantly lower than a mean pre-treatment miR-122 blood level in HCV patients who rebound after the 12-week treatment. Also preferably, the treatment lasts 12 weeks, and the patient's pre-treatment miR-122 level in blood is at least 1-fold lower than a mean pre-treatment miR-122 blood level in HCV patients who rebound after the 12-week treatment. Also preferably, the treatment lasts 12 weeks, and the patient's pre-treatment miR-122 level in blood is at least 2-fold lower than a mean pre-treatment miR-122 blood level in HCV patients who rebound after the 12-week treatment. Also preferably, the treatment lasts 12 weeks, and the patient's pre-treatment miR-122 level in blood is at least 3-fold lower than a mean pre-treatment miR-122 blood level in HCV patients who rebound after the 12-week treatment. Also preferably, the treatment lasts 12 weeks, and the patient's pre-treatment miR-122 level in blood is at least 4-fold lower than a mean pre-treatment miR-122 blood level in HCV patients who rebound after the 12-week treatment. Also preferably, the treatment lasts 12 weeks, and the patient's pre-treatment miR-122 level in blood is from 1-fold to 4-fold lower than a mean pre-treatment miR-122 blood level in HCV patients who rebound after the 12-week treatment. For example, the direct-acting antiviral regimen can comprise GS-7977 dosed at 400 mg QD, daclatasvir dosed at 60 mg QD, and, optionally, ribavirin dosed once or twice daily. The regimen can be administered for a duration of 8 to 24 weeks, including for example, 12 weeks. The present technology also features the same aspect of the invention as described immediately above, except that pre-treatment miR-21 level in blood is used in lieu of pre-treatment miR-122 level in blood.

In another embodiment, the present invention features methods of treating HCV comprising administering to a patient in need thereof a direct-acting antiviral regimen, wherein the patient is tested for miR-122 level in blood prior to the treatment. The direct-acting antiviral regimen comprises a combination of asunaprevir and daclatasvir. Preferably, the treatment lasts 12 weeks, and the patient's pre-treatment miR-122 level in blood is significantly lower than a mean pre-treatment miR-122 blood level in HCV patients who rebound after the 12-week treatment. Also preferably, the treatment lasts 12 weeks, and the patient's pre-treatment miR-122 level in blood is at least 1-fold lower than a mean pre-treatment miR-122 blood level in HCV patients who rebound after the 12-week treatment. Also preferably, the treatment lasts 12 weeks, and the patient's pre-treatment miR-122 level in blood is at least 2-fold lower than a mean pre-treatment miR-122 blood level in HCV patients who rebound after the 12-week treatment. Also preferably, the treatment lasts 12 weeks, and the patient's pre-treatment miR-122 level in blood is at least 3-fold lower than a mean pre-treatment miR-122 blood level in HCV patients who rebound after the 12-week treatment. Also preferably, the treatment lasts 12 weeks, and the patient's pre-treatment miR-122 level in blood is at least 4-fold lower than a mean pre-treatment miR-122 blood level in HCV patients who rebound after the 12-week treatment. Also preferably, the treatment lasts 12 weeks, and the patient's pre-treatment miR-122 level in blood is from 1-fold to 4-fold lower than a mean pre-treatment miR-122 blood level in HCV patients who rebound after the 12-week treatment. For example, the direct-acting antiviral regimen can comprise asunaprevir dosed at 100 mg QD or 200 mg BID, daclatasvir dosed at 60 mg QD, and, optionally, ribavirin dosed once or twice daily. The regimen can be administered for a duration of 8 to 24 weeks, including for example, 12 weeks. The present technology also features the same aspect of the invention as described immediately above, except that pre-treatment miR-21 level in blood is used in lieu of pre-treatment miR-122 level in blood.

In another embodiment, the present invention features methods of treating HCV comprising administering to a patient in need thereof a direct-acting antiviral regimen, wherein the patient is tested for miR-122 level in blood prior to the treatment. The direct-acting antiviral regimen comprises a combination of asunaprevir, daclatasvir, and BMS-791325. Preferably, the treatment lasts 12 weeks, and the patient's pre-treatment miR-122 level in blood is significantly lower than a mean pre-treatment miR-122 blood level in HCV patients who rebound after the 12-week treatment. Also preferably, the treatment lasts 12 weeks, and the patient's pre-treatment miR-122 level in blood is at least 1-fold lower than a mean pre-treatment miR-122 blood level in HCV patients who rebound after the 12-week treatment. Also preferably, the treatment lasts 12 weeks, and the patient's pre-treatment miR-122 level in blood is at least 2-fold lower than a mean pre-treatment miR-122 blood level in HCV patients who rebound after the 12-week treatment. Also preferably, the treatment lasts 12 weeks, and the patient's pre-treatment miR-122 level in blood is at least 3-fold lower than a mean pre-treatment miR-122 blood level in HCV patients who rebound after the 12-week treatment. Also preferably, the treatment lasts 12 weeks, and the patient's pre-treatment miR-122 level in blood is at least 4-fold lower than a mean pre-treatment miR-122 blood level in HCV patients who rebound after the 12-week treatment. Also preferably, the treatment lasts 12 weeks, and the patient's pre-treatment miR-122 level in blood is from 1-fold to 4-fold lower than a mean pre-treatment miR-122 blood level in HCV patients who rebound after the 12-week treatment. For example, the direct-acting antiviral regimen can comprise asunaprevir dosed at 200 mg twice daily, daclatasvir dosed at 30 mg twice daily, and BMS-791325 dosed at 75 or 150 mg twice daily. The regimen can be administered for a duration of 8 to 24 weeks, including for example, 12 or 24 weeks. The present technology also features the same aspect of the invention as described immediately above, except that pre-treatment miR-21 level in blood is used in lieu of pre-treatment miR-122 level in blood.

In another embodiment, the present invention features methods of treating HCV comprising administering to a patient in need thereof a direct-acting antiviral regimen, wherein the patient is tested for miR-122 level in blood prior to the treatment. The direct-acting antiviral regimen comprises a combination of GS-7977 and simeprevir. Preferably, the treatment lasts 12 weeks, and the patient's pre-treatment miR-122 level in blood is significantly lower than a mean pre-treatment miR-122 blood level in HCV patients who rebound after the 12-week treatment. Also preferably, the treatment lasts 12 weeks, and the patient's pre-treatment miR-122 level in blood is at least 1-fold lower than a mean pre-treatment miR-122 blood level in HCV patients who rebound after the 12-week treatment. Also preferably, the treatment lasts 12 weeks, and the patient's pre-treatment miR-122 level in blood is at least 2-fold lower than a mean pre-treatment miR-122 blood level in HCV patients who rebound after the 12-week treatment. Also preferably, the treatment lasts 12 weeks, and the patient's pre-treatment miR-122 level in blood is at least 3-fold lower than a mean pre-treatment miR-122 blood level in HCV patients who rebound after the 12-week treatment. Also preferably, the treatment lasts 12 weeks, and the patient's pre-treatment miR-122 level in blood is at least 4-fold lower than a mean pre-treatment miR-122 blood level in HCV patients who rebound after the 12-week treatment. Also preferably, the treatment lasts 12 weeks, and the patient's pre-treatment miR-122 level in blood is from 1-fold to 4-fold lower than a mean pre-treatment miR-122 blood level in HCV patients who rebound after the 12-week treatment. For example, the direct-acting antiviral regimen can comprise GS-7977 dosed at 400 mg QD, simeprevir dosed at 150 mg QD, and, optionally, ribavirin dosed once or twice daily. The regimen can be administered for a duration of 8 to 24 weeks, including for example, 12 weeks. The present technology also features the same aspect of the invention as described immediately above, except that pre-treatment miR-21 level in blood is used in lieu of pre-treatment miR-122 level in blood.

In another embodiment, the present invention features methods of treating HCV comprising administering to a patient in need thereof a direct-acting antiviral regimen, wherein the patient is tested for miR-122 level in blood prior to the treatment. The direct-acting antiviral regimen comprises a combination of daclatasvir and simeprevir. Preferably, the treatment lasts 12 weeks, and the patient's pre-treatment miR-122 level in blood is significantly lower than a mean pre-treatment miR-122 blood level in HCV patients who rebound after the 12-week treatment. Also preferably, the treatment lasts 12 weeks, and the patient's pre-treatment miR-122 level in blood is at least 1-fold lower than a mean pre-treatment miR-122 blood level in HCV patients who rebound after the 12-week treatment. Also preferably, the treatment lasts 12 weeks, and the patient's pre-treatment miR-122 level in blood is at least 2-fold lower than a mean pre-treatment miR-122 blood level in HCV patients who rebound after the 12-week treatment. Also preferably, the treatment lasts 12 weeks, and the patient's pre-treatment miR-122 level in blood is at least 3-fold lower than a mean pre-treatment miR-122 blood level in HCV patients who rebound after the 12-week treatment. Also preferably, the treatment lasts 12 weeks, and the patient's pre-treatment miR-122 level in blood is at least 4-fold lower than a mean pre-treatment miR-122 blood level in HCV patients who rebound after the 12-week treatment. Also preferably, the treatment lasts 12 weeks, and the patient's pre-treatment miR-122 level in blood is from 1-fold to 4-fold lower than a mean pre-treatment miR-122 blood level in HCV patients who rebound after the 12-week treatment. For example, the direct-acting antiviral regimen can comprise daclatasvir dosed at 30 mg QD, simeprevir dosed at 150 mg QD, and, optionally, ribavirin dosed once or twice daily. The regimen can be administered for a duration of 8 to 24 weeks, including for example, 12 weeks. The present technology also features the same aspect of the invention as described immediately above, except that pre-treatment miR-21 level in blood is used in lieu of pre-treatment miR-122 level in blood.

In another embodiment, the present invention features methods of treating HCV comprising administering to a patient in need thereof a direct-acting antiviral regimen, wherein the patient is tested for miR-122 level in blood prior to the treatment. The direct-acting antiviral regimen comprises a combination of simeprevir and TMC647055. Simeprevir can be co-administered or co-formulated with an inhibitor of cytochrome P-450 (such as ritonavir). Preferably, the treatment lasts 12 weeks, and the patient's pre-treatment miR-122 level in blood is significantly lower than a mean pre-treatment miR-122 blood level in HCV patients who rebound after the 12-week treatment. Also preferably, the treatment lasts 12 weeks, and the patient's pre-treatment miR-122 level in blood is at least 1-fold lower than a mean pre-treatment miR-122 blood level in HCV patients who rebound after the 12-week treatment. Also preferably, the treatment lasts 12 weeks, and the patient's pre-treatment miR-122 level in blood is at least 2-fold lower than a mean pre-treatment miR-122 blood level in HCV patients who rebound after the 12-week treatment. Also preferably, the treatment lasts 12 weeks, and the patient's pre-treatment miR-122 level in blood is at least 3-fold lower than a mean pre-treatment miR-122 blood level in HCV patients who rebound after the 12-week treatment. Also preferably, the treatment lasts 12 weeks, and the patient's pre-treatment miR-122 level in blood is at least 4-fold lower than a mean pre-treatment miR-122 blood level in HCV patients who rebound after the 12-week treatment. Also preferably, the treatment lasts 12 weeks, and the patient's pre-treatment miR-122 level in blood is from 1-fold to 4-fold lower than a mean pre-treatment miR-122 blood level in HCV patients who rebound after the 12-week treatment. For example, the direct-acting antiviral regimen can comprise simeprevir dosed at 150 mg QD, TMC647055 dosed at 500 or 1000 mg BID, ritonavir dosed at 100 mg QD, and, optionally, ribavirin dosed once or twice daily. The regimen can be administered for a duration of 8 to 24 weeks, including for example, 12 weeks. The present technology also features the same aspect of the invention as described immediately above, except that pre-treatment miR-21 level in blood is used in lieu of pre-treatment miR-122 level in blood.

In another embodiment, the direct-acting antiviral regimen comprises a combination of danoprevir and mericitabine. Danoprevir can be co-administered or co-formulated with an inhibitor of cytochrome P-450 (such as ritonavir). Preferably, the treatment lasts 12 weeks, and the patient's pre-treatment miR-122 level in blood is significantly lower than a mean pre-treatment miR-122 blood level in HCV patients who rebound after the 12-week treatment. Also preferably, the treatment lasts 12 weeks, and the patient's pre-treatment miR-122 level in blood is at least 1-fold lower than a mean pre-treatment miR-122 blood level in HCV patients who rebound after the 12-week treatment. Also preferably, the treatment lasts 12 weeks, and the patient's pre-treatment miR-122 level in blood is at least 2-fold lower than a mean pre-treatment miR-122 blood level in HCV patients who rebound after the 12-week treatment. Also preferably, the treatment lasts 12 weeks, and the patient's pre-treatment miR-122 level in blood is at least 3-fold lower than a mean pre-treatment miR-122 blood level in HCV patients who rebound after the 12-week treatment. Also preferably, the treatment lasts 12 weeks, and the patient's pre-treatment miR-122 level in blood is at least 4-fold lower than a mean pre-treatment miR-122 blood level in HCV patients who rebound after the 12-week treatment. Also preferably, the treatment lasts 12 weeks, and the patient's pre-treatment miR-122 level in blood is from 1-fold to 4-fold lower than a mean pre-treatment miR-122 blood level in HCV patients who rebound after the 12-week treatment. For example, the direct-acting antiviral regimen can comprise danoprevir dosed at 100 mg BID, mericitabine dosed at 1000 mg BID, ritonavir dosed at 100 mg QD, and, optionally, ribavirin dosed once or twice daily. The regimen can be administered for a duration of 8 to 24 weeks, including for example, 12 weeks. The present technology also features the same aspect of the invention as described immediately above, except that pre-treatment miR-21 level in blood is used in lieu of pre-treatment miR-122 level in blood.

In another embodiment, the present invention features methods of treating HCV comprising administering to a patient in need thereof a direct-acting antiviral regimen, wherein the patient is tested for miR-122 level in blood prior to the treatment. The direct-acting antiviral regimen comprises a combination of danoprevir, mericitabine, and setrobuvir. Danoprevir can be co-administered or co-formulated with an inhibitor of cytochrome P-450 (such as ritonavir). Preferably, the treatment lasts 12 weeks, and the patient's pre-treatment miR-122 level in blood is significantly lower than a mean pre-treatment miR-122 blood level in HCV patients who rebound after the 12-week treatment. Also preferably, the treatment lasts 12 weeks, and the patient's pre-treatment miR-122 level in blood is at least 1-fold lower than a mean pre-treatment miR-122 blood level in HCV patients who rebound after the 12-week treatment. Also preferably, the treatment lasts 12 weeks, and the patient's pre-treatment miR-122 level in blood is at least 2-fold lower than a mean pre-treatment miR-122 blood level in HCV patients who rebound after the 12-week treatment. Also preferably, the treatment lasts 12 weeks, and the patient's pre-treatment miR-122 level in blood is at least 3-fold lower than a mean pre-treatment miR-122 blood level in HCV patients who rebound after the 12-week treatment. Also preferably, the treatment lasts 12 weeks, and the patient's pre-treatment miR-122 level in blood is at least 4-fold lower than a mean pre-treatment miR-122 blood level in HCV patients who rebound after the 12-week treatment. Also preferably, the treatment lasts 12 weeks, and the patient's pre-treatment miR-122 level in blood is from 1-fold to 4-fold lower than a mean pre-treatment miR-122 blood level in HCV patients who rebound after the 12-week treatment. For example, the direct-acting antiviral regimen can comprise danoprevir dosed at 100 mg BID, mericitabine dosed at 500 mg BID, setrobuvir dosed at 200 mg BID, ritonavir dosed at 100 mg QD, and, optionally, ribavirin dosed once or twice daily. The regimen can be administered for a duration of 8 to 24 weeks, including for example, 12 weeks. The present technology also features the same aspect of the invention as described immediately above, except that pre-treatment miR-21 level in blood is used in lieu of pre-treatment miR-122 level in blood.

In another embodiment, the present invention features methods of treating HCV comprising administering to a patient in need thereof a direct-acting antiviral regimen, wherein the patient is tested for miR-122 level in blood prior to the treatment. The direct-acting antiviral regimen comprises a combination of danoprevir and setrobuvir. Danoprevir can be co-administered or co-formulated with an inhibitor of cytochrome P-450 (such as ritonavir). For example, the direct-acting antiviral regimen can comprise danoprevir dosed at 100 mg BID, setrobuvir dosed at 200 mg BID, ritonavir dosed at 100 mg QD, and, optionally, ribavirin dosed once or twice daily. The regimen can be administered for a duration of 8 to 24 weeks, including for example, 12 weeks. The present technology also features the same aspect of the invention as described immediately above, except that pre-treatment miR-21 level in blood is used in lieu of pre-treatment miR-122 level in blood.

In another embodiment, the present invention features methods of treating HCV comprising administering to a patient in need thereof a direct-acting antiviral regimen, wherein the patient is tested for miR-122 level in blood prior to the treatment. The direct-acting antiviral regimen comprises a combination of BI201335 and BI207127. Preferably, the treatment lasts 12 weeks, and the patient's pre-treatment miR-122 level in blood is significantly lower than a mean pre-treatment miR-122 blood level in HCV patients who rebound after the 12-week treatment. Also preferably, the treatment lasts 12 weeks, and the patient's pre-treatment miR-122 level in blood is at least 1-fold lower than a mean pre-treatment miR-122 blood level in HCV patients who rebound after the 12-week treatment. Also preferably, the treatment lasts 12 weeks, and the patient's pre-treatment miR-122 level in blood is at least 2-fold lower than a mean pre-treatment miR-122 blood level in HCV patients who rebound after the 12-week treatment. Also preferably, the treatment lasts 12 weeks, and the patient's pre-treatment miR-122 level in blood is at least 3-fold lower than a mean pre-treatment miR-122 blood level in HCV patients who rebound after the 12-week treatment. Also preferably, the treatment lasts 12 weeks, and the patient's pre-treatment miR-122 level in blood is at least 4-fold lower than a mean pre-treatment miR-122 blood level in HCV patients who rebound after the 12-week treatment. Also preferably, the treatment lasts 12 weeks, and the patient's pre-treatment miR-122 level in blood is from 1-fold to 4-fold lower than a mean pre-treatment miR-122 blood level in HCV patients who rebound after the 12-week treatment. For example, the direct-acting antiviral regimen can comprise BI201335 dosed at 120 mg QD, BI207127 dosed at 600 mg BID or TID, and, optionally, ribavirin dosed once or twice daily. The regimen can be administered for a duration of 8 to 40 weeks, including for example, 12 weeks, 16 weeks, 24 weeks, and 28 weeks. The present technology also features the same aspect of the invention as described immediately above, except that pre-treatment miR-21 level in blood is used in lieu of pre-treatment miR-122 level in blood.

In another embodiment, the direct-acting antiviral regimen comprises a combination of telaprevir and VX222. Preferably, the treatment lasts 12 weeks, and the patient's pre-treatment miR-122 level in blood is significantly lower than a mean pre-treatment miR-122 blood level in HCV patients who rebound after the 12-week treatment. Also preferably, the treatment lasts 12 weeks, and the patient's pre-treatment miR-122 level in blood is at least 1-fold lower than a mean pre-treatment miR-122 blood level in HCV patients who rebound after the 12-week treatment. Also preferably, the treatment lasts 12 weeks, and the patient's pre-treatment miR-122 level in blood is at least 2-fold lower than a mean pre-treatment miR-122 blood level in HCV patients who rebound after the 12-week treatment. Also preferably, the treatment lasts 12 weeks, and the patient's pre-treatment miR-122 level in blood is at least 3-fold lower than a mean pre-treatment miR-122 blood level in HCV patients who rebound after the 12-week treatment. Also preferably, the treatment lasts 12 weeks, and the patient's pre-treatment miR-122 level in blood is at least 4-fold lower than a mean pre-treatment miR-122 blood level in HCV patients who rebound after the 12-week treatment. Also preferably, the treatment lasts 12 weeks, and the patient's pre-treatment miR-122 level in blood is from 1-fold to 4-fold lower than a mean pre-treatment miR-122 blood level in HCV patients who rebound after the 12-week treatment. For example, the direct-acting antiviral regimen can comprise telaprevir dosed at 1125 mg BID, VX222 dosed at 100 mg BID, and, optionally, ribavirin dosed once or twice daily. The regimen can be administered for a duration of 8 to 24 weeks, including for example, 12 weeks. The present technology also features the same aspect of the invention as described immediately above, except that pre-treatment miR-21 level in blood is used in lieu of pre-treatment miR-122 level in blood.

In another embodiment, the present invention features methods of treating HCV comprising administering to a patient in need thereof a direct-acting antiviral regimen, wherein the patient is tested for miR-122 level in blood prior to the treatment. The direct-acting antiviral regimen comprises a combination of telaprevir and ALS2200. Preferably, the treatment lasts 12 weeks, and the patient's pre-treatment miR-122 level in blood is significantly lower than a mean pre-treatment miR-122 blood level in HCV patients who rebound after the 12-week treatment. Also preferably, the treatment lasts 12 weeks, and the patient's pre-treatment miR-122 level in blood is at least 1-fold lower than a mean pre-treatment miR-122 blood level in HCV patients who rebound after the 12-week treatment. Also preferably, the treatment lasts 12 weeks, and the patient's pre-treatment miR-122 level in blood is at least 2-fold lower than a mean pre-treatment miR-122 blood level in HCV patients who rebound after the 12-week treatment. Also preferably, the treatment lasts 12 weeks, and the patient's pre-treatment miR-122 level in blood is at least 3-fold lower than a mean pre-treatment miR-122 blood level in HCV patients who rebound after the 12-week treatment. Also preferably, the treatment lasts 12 weeks, and the patient's pre-treatment miR-122 level in blood is at least 4-fold lower than a mean pre-treatment miR-122 blood level in HCV patients who rebound after the 12-week treatment. Also preferably, the treatment lasts 12 weeks, and the patient's pre-treatment miR-122 level in blood is from 1-fold to 4-fold lower than a mean pre-treatment miR-122 blood level in HCV patients who rebound after the 12-week treatment. For example, the direct-acting antiviral regimen can comprise telaprevir dosed at 1125 mg BID, ALS2200 dosed at 50 mg QD, 100 mg QD, or 200 mg QD, and, optionally, ribavirin dosed once or twice daily. The regimen can be administered for a duration of 8 to 24 weeks, including for example, 12 weeks. The present technology also features the same aspect of the invention as described immediately above, except that pre-treatment miR-21 level in blood is used in lieu of pre-treatment miR-122 level in blood.

Certain aspects of the present invention comprise treatment of an HCV-infected patient with a direct-acting antiviral regimen. The patient being treated can be a treatment naïve patient, a treatment experienced patient, including, but not limited to, a relapser, an interferon partial responder, an interferon non-responder (e.g., a null responder), or a patient unable to take interferon. The patient may be infected with, for example and without limitation, HCV genotype 1, such as HCV genotype la or HCV genotype 1b; or HCV genotype 2 or 3. The treatment may also be effective against other HCV genotypes.

In some of the methods described herein, it is desirable to identify miRNAs present in a sample. Detection and quantification of miRNA expression can be achieved by any one of a number of methods well known in the art. Using the known sequences for miRNAs, such as miR-122 and miR-21, specific probes and primers can be designed for use in the detection methods described below as appropriate.

Preferably, the sample is a body tissue or body fluid obtained from a human subject (e.g., an HCV-infected patient). MicroRNAs have been shown to be present in various body fluids such as serum, plasma, cerebrospinal fluid, saliva, and urine. In one embodiment, the sample is a serum sample.

In some cases, RNA detection requires isolation of nucleic acid from a biological sample, such as a cell, biological fluid, or tissue sample. Nucleic acids, including RNA and specifically miRNA, can be isolated using any suitable technique known in the art. For example, phenol-based extraction is a common method for isolation of RNA. In addition, extraction procedures such as those using Qiagen miRNeasy® Mini Kit are efficient methods for isolating total RNA, including microRNA, from a biological sample.

In some embodiments, the miRNA expression level can be determined by reverse transcription of miRNA, followed by amplification of the reverse-transcribed miRNA by polymerase chain reaction (RT-PCR). The miRNA expression level can be quantified in comparison with an internal standard, for example, the level of mRNA from a “housekeeping” gene present in the same sample. Suitable “housekeeping” genes for use as an internal standard include, for example, beta-actin, myosin heavy chain, or glyceraldehyde-3-phosphate dehydrogenase (GAPDH). The methods for quantitative RT-PCR and variations thereof are within the level of skill in the art. Any method of PCR that can determine the expression of a nucleic acid molecule, including an miRNA, falls within the scope of the present disclosure.

In some embodiments, the miRNA expression level can be determined by quantitative RT-PCR (qRT-PCR). qRT-PCR is a type of PCR used to rapidly measure the quantity of a product of the PCR. qRT-PCR is commonly used for the purpose of determining whether a nucleic acid sequence, such as a miRNA, is present in a sample, and if it is present, the number of copies in the sample. qRT-PCR methods allowing for real-time analysis utilize, for example, SYBR Green (a double stranded DNA dye) or a fluorescent reporter probe.

In some embodiments, the miRNA expression level can be determined using a microarray. A nucleic acid microarray is a microscopic, ordered array of nucleic acids that enables parallel analysis of several nucleic acids. A DNA microarray consists of different nucleic acid probes, known as capture probes that are chemically attached to a solid substrate, which can be a microchip, a glass slide, or a microsphere-sized bead. DNA microarrays can be used, for example, to measure the expression levels of large numbers of miRNAs simultaneously. Microarray analysis of miRNAs can be accomplished according to any method known in the art.

In some embodiments, the miRNA can be detected by in situ hybridization (ISH). ISH is a technology that allows detection of specific nucleic acid sequences, such as microRNAs, in tissue samples at the cellular level. ISH can be combined with cytochemistry, immunocytochemistry and immunohistochemistry to localize sequences to specific cells within populations, such as tissues and blood samples. ISH procedures such as those using miRCURY LNA™ microRNA ISH Optimization Kit (Exiqon) can be used to detect miRNAs in a portion or section of a cell or tissue.

In some embodiments, the miRNA expression level can be determined using next-generation, or second generation, sequencing technology. Second generation sequencing technology is compared to first generation, or capillary electrophoresis (CE)-based Sanger, sequencing technology. In sequencing technologies, the bases of a small fragment of DNA are sequentially identified from signals emitted as each fragment is re-synthesized from a DNA template strand. Second generation sequencing extends this process across millions of reactions in a massively parallel fashion, rather than being limited to a single or a few DNA fragments.

In some embodiments, the miRNA expression level in a sample obtained from a subject is compared to a control. The miRNA expression level in a sample obtained from a subject can be compared with the miRNA expression level of a control sample. Cells or biological samples obtained from one or more cells, tissues, or organisms, as described above, can comprise suitable controls for the quantitative methods set forth herein. In another aspect, the relative miRNA expression level in a sample obtained from a subject can be determined with respect to one or more RNA expression standards. The standards can comprise, for example, a zero miRNA expression level, the miRNA expression level in a standard cell line, or the mean miRNA expression level obtained for a population of human controls.

An aspect of the present invention provides methods to prevent or control proliferation of strains of Hepatitis C virus that are resistant to treatment with a direct-acting antiviral regimen consisting of a protease inhibitor, a non-nucleoside polymerase inhibitor, and ribavirin, the method comprising: identifying a patient having an miR-122 or miR-21 expression level that is predictive of an inadequate response to the direct-acting antiviral regimen; and refraining from administering the direct-acting antiviral regimen to the identified patient. In certain embodiments, the microRNA is miR-122 or miR-21. The miR-122 or miR-21 expression level can be determined by measuring circulating miR-122 or miR-21 in plasma or serum samples obtained from the patient infected with Hepatitis C virus. An expression level that is predictive of an inadequate response to the direct-acting antiviral regimen can be an expression level that is greater than a pre-determined control level. In some embodiments, an expression level that is predictive of an inadequate response to the direct-acting antiviral regimen can be an expression level that is at least 1-fold greater than a pre-determined control level. In some embodiments, an expression level that is predictive of an inadequate response to the direct-acting antiviral regimen can be an expression level that is at least 2-fold greater than a pre-determined control level. In some embodiments, an expression level that is predictive of an inadequate response to the direct-acting antiviral regimen can be an expression level that is at least 3-fold greater than a pre-determined control level. In some embodiments, an expression level that is predictive of an inadequate response to the direct-acting antiviral regimen can be an expression level that is at least 4-fold greater than a pre-determined control level. The pre-determined control level can be a range or a specific value. In some embodiments, an expression level that is predictive of an inadequate response to the direct-acting antiviral regimen can be an expression level that is from 1-fold to 4-fold greater than a pre-determined control level. The pre-determined control level can be determined from empirical data, such as, by obtaining a mean expression level from a population of subjects. The population of subjects can be a population of healthy subjects or a population of HCV-infected subjects.

An aspect of the present invention provides methods to monitor responsiveness of Hepatitis C virus to treatment with a direct-acting antiviral regimen. The methods comprise assessing microRNA expression in a patient infected with Hepatitis C virus prior to administration of the direct-acting antiviral regimen to establish a baseline expression level; administering the direct-acting antiviral regimen to the patient; and re-assessing microRNA expression in the patient after the administration of the direct-acting antiviral regimen to establish an on-treatment expression level. In certain embodiments, the microRNA is miR-122 or miR-21. The miR-122 or miR-21 expression level can be determined by measuring circulating miR-122 or miR-21 in plasma or serum samples obtained from the patient infected with Hepatitis C virus. The methods can further comprise the step of discontinuing the direct-acting antiviral regimen where the on-treatment expression level is equal to or greater than the baseline expression level. Alternatively, the methods can further comprise the step of continuing the direct-acting antiviral regimen where the on-treatment expression level is less than the baseline expression level. For example, the methods can further comprise the step of continuing the direct-acting antiviral regimen where the on-treatment expression level is at least four-fold less than the baseline expression level.

In further aspects, the present invention provides an article of manufacture comprising: packaging material containing a composition effective to treat a Hepatitis C virus infection; and a label indicating that the composition can be used to treat Hepatitis C virus infection in a patient having an miR-122 or miR-21 expression level that is predictive of a sustained response to the composition. For example, the label can indicate that the composition can be used to treat Hepatitis C virus infection in a patient having an miR-122 or miR-21 expression level that is equal to or less than a pre-determined control level. In some embodiments, the label can indicate that the composition can be used to treat Hepatitis C virus infection in a patient having an miR-122 or miR-21 expression level that is at least 1-fold less than a pre-determined control level. In some embodiments, the label can indicate that the composition can be used to treat Hepatitis C virus infection in a patient having an miR-122 or miR-21 expression level that is at least 2-fold less than a pre-determined control level. In some embodiments, the label can indicate that the composition can be used to treat Hepatitis C virus infection in a patient having an miR-122 or miR-21 expression level that is at least 3-fold less than a pre-determined control level. In some embodiments, the label can indicate that the composition can be used to treat Hepatitis C virus infection in a patient having an miR-122 or miR-21 expression level that is at least 4-fold less than a pre-determined control level. The pre-determined control level can be a range or a specific value. In some embodiments, the label can indicate that the composition can be used to treat Hepatitis C virus infection in a patient having an miR-122 or miR-21 expression level that is from 1-fold to 4-fold less than a pre-determined control level. The pre-determined control level can be determined from empirical data, such as, by obtaining a mean expression level from a population of subjects. The population of subjects can be a population of healthy subjects or a population of HCV-infected subjects. Particular compositions effective to treat a Hepatitis C virus infection include, for example, protease inhibitors, nucleoside or nucleotide polymerase inhibitors, non-nucleoside polymerase inhibitors, NS3B inhibitors, NS4A inhibitors, NS5A inhibitors, NS5B inhibitors, cyclophilin inhibitors, and combinations of any of the foregoing. For example, a composition effective to treat a Hepatitis C virus infection can comprise therapeutic agent 1, therapeutic agent 2, or a combination thereof. The composition can be co-administered or co-formulated with ritonavir. The composition also can be co-administered or co-formulated with ribavirin.

In still further aspects, the present invention provides an article of manufacture comprising: packaging material containing a composition effective to treat a Hepatitis C virus infection; and a label indicating that the composition should not be used to treat Hepatitis C virus infection in a patient having an miR-122 or miR-21 expression level that is predictive of an inadequate response to the composition. For example, the label can indicate that the composition should not be used to treat Hepatitis C virus infection in a patient having an miR-122 or miR-21 expression level that is greater than a pre-determined control level. In some embodiments, the label can indicate that the composition can be used to treat Hepatitis C virus infection in a patient having an miR-122 or miR-21 expression level that is at least 1-fold greater than a pre-determined control level. In some embodiments, the label can indicate that the composition can be used to treat Hepatitis C virus infection in a patient having an miR-122 or miR-21 expression level that is at least 2-fold greater than a pre-determined control level. In some embodiments, the label can indicate that the composition can be used to treat Hepatitis C virus infection in a patient having an miR-122 or miR-21 expression level that is at least 3-fold greater than a pre-determined control level. In some embodiments, the label can indicate that the composition can be used to treat Hepatitis C virus infection in a patient having an miR-122 or miR-21 expression level that is at least 4-fold greater than a pre-determined control level. In some embodiments, the label can indicate that the composition can be used to treat Hepatitis C virus infection in a patient having an miR-122 or miR-21 expression level that is from 1-fold to 4-fold greater than a pre-determined control level. The pre-determined control level can be a range or a specific value. The pre-determined control level can be determined from empirical data, such as, by obtaining a mean expression level from a population of subjects. The population of subjects can be a population of healthy subjects or a population of HCV-infected subjects. Particular compositions effective to treat a Hepatitis C virus infection include, for example, protease inhibitors, nucleoside or nucleotide polymerase inhibitors, non-nucleoside polymerase inhibitors, NS3B inhibitors, NS4A inhibitors, NS5A inhibitors, NS5B inhibitors, cyclophilin inhibitors, and combinations of any of the foregoing. For example, a composition effective to treat a Hepatitis C virus infection can comprise therapeutic agent 1, therapeutic agent 2, or a combination thereof. The composition can be co-administered or co-formulated with ritonavir. The composition also can be co-administered or co-formulated with ribavirin.

In yet further aspects, the present invention provides systems for effective treatment of Hepatitis C virus infection, comprising: a measurement of an miR-122 or miR-12 expression level for a patient; and a direct-acting antiviral regimen. The direct-acting antiviral regimen can comprise an HCV protease inhibitor and an HCV polymerase inhibitor. The systems can further comprise an assay for providing the measurement of the miR-122 or miR-21 expression level. The systems also can comprise directions whether to administer the direct-acting antiviral regimen to a patient based on the measurement.

### EXAMPLE 1

### Use of Direct-Acting Antiviral Regimen with Ribavirin to Treat Treatment-Naïve or Non-Responder Subjects Infected with HCV Genotype 1

Group A. Previously untreated subjects having HCV infection were treated with a protease inhibitor (in combination with ritonavir), a polymerase inhibitor, and ribavirin. The treatment was without interferon.

Subjects included 14 treatment naïve subjects between the ages of 18 and 65. One subject discontinued the study at week 1. Therefore, a total of 13 subjects were under study. All of the thirteen subjects completed 12 weeks of therapy with a direct-acting antiviral regimen comprising Compound 1/r dosed in combination with Compound 2 and RBV. Compound 1 (150 mg QD) was dosed with 100 mg QD ritonavir, 400 mg BID Compound 2, and RBV in treatment naïve subjects infected with GT1 HCV.

Group B. Peginterferon +ribavirin (P/RBV) treatment-experienced patients were treated with a direct-acting antiviral regimen comprising a protease inhibitor (in combination with ritonavir), a polymerase inhibitor, and ribavirin. The treatment was without interferon.

Subjects included 17 P/RBV treatment-experienced patients between the ages of 18 and 65. Subjects were treated with Compound 1/r dosed in combination with Compound 2 and RBV for 12 weeks. Compound 1 (150 mg QD) was dosed with 100 mg QD ritonavir, 400 mg BID Compound 2, and RBV in P/RBV treatment-experienced patients infected with GT1 HCV. During the treatment, four patients had breakthroughs and discontinued the study before week 7.

The characteristics of the patients prior to initiating the direct-acting antiviral regimen are shown in the table below.

TABLE 1

| | Group 2 | Group 3 |
|---|---|---|
| Genotype (1a/1b) | 11/3 | 16/1 |
| IL28B: | | |
| CC | 5 | 0 |
| CT | 7 | 11 |
| TT | 2 | 5 |
| Undetermined | 0 | 1 |
| Median baseline HCV RNA | 6.9 [3.1-7.5] | 6.9 [6.0-7.8] |
| (log IU/mL) | | |

Expression profiling of circulating microRNA in serum was performed to identify baseline microRNA levels as well as changes in microRNA that occur as a result of treatment.

Sample preparation and microRNA real-time PCR. Serum samples were collected at baseline (“BASE”), week 2 on-treatment (“W2”), week 10 on-treatment (“W10”), and 8 weeks after completing the 12 week regimen (“Post Week 8”) and frozen until analysis. Total RNA was extracted from serum using the Qiagen miRNeasy® Mini Kit. Serum was thawed on ice and centrifuged at 3000×g for 5 min in a 4° C. microcentrifuge. An aliquot of 200 μL of serum per sample was transferred to a new microcentrifuge tube and 750 μl of a Qiazol mixture containing 1.25 μg/mL of MS2 bacteriophage RNA was added to the serum. The tube was mixed and incubated for 5 min followed by the addition of 200 μL chloroform. The tube was mixed, incubated for 2 min and centrifuged at 12,000×g for 15 min in a 4° C. microcentrifuge. The upper aqueous phase was transferred to a new microcentrifuge tube and 1.5 volume of 100% ethanol was added. The contents were mixed thoroughly and 750 μL of the sample was transferred to a Qiagen RNeasy® Mini spin column in a collection tube followed by centrifugation at 15,000×g for 30 sec at room temperature. The process was repeated until all remaining sample had been loaded. The Qiagen RNeasy® Mini spin column was rinsed with 700 μL Qiagen RWT buffer and centrifuged at 15,000×g for 1 min at room temperature followed by another rinse with 500 μL Qiagen RPE buffer and centrifuged at 15,000×g for 1 min at room temperature. A rinse step (500 μL Qiagen RPE buffer) was repeated 2×. The Qiagen RNeasy® Mini spin column was transferred to a new collection tube and centrifuged at 15,000×g for 2 min at room temperature. The Qiagen RNeasy® Mini spin column was transferred to a new microcentrifuge tube and the lid was left uncapped for 1 min to allow the column to dry. Total RNA was eluted by adding 50 μL of RNase-free water to the membrane of the Qiagen RNeasy® mini spin column and incubating for 1 min before centrifugation at 15,000×g for 1 min at room temperature. The RNA was stored in a −80° C. freezer.

15 μl RNA was reverse transcribed in 75 μl reactions using the miRCURY LNA™ Universal RT microRNA PCR, Polyadenylation and cDNA synthesis kit (Exiqon). cDNA was diluted 50× and assayed in 10 ul PCR reactions according to the protocol for miRCURY LNA™ Universal RT microRNA PCR; each microRNA was assayed once by qPCR on the microRNA Ready-to-Use PCR, Human panel I and panel II. Negative controls excluding template from the reverse transcription reaction was performed and profiled like the samples. The amplification was performed in a LightCycler® 480 Real-Time PCR System (Roche) in 384 well plates. The amplification curves were analyzed using the Roche LC software, both for determination of Cp (by the 2nd derivative method) and for melting curve analysis. All data was normalized to the average of assays detected in all samples (average—assay Cp). In PCR reactions, each cycle represents a doubling of amplicon. Cp is the cycle where detection for a given sample reaches the threshold. Delta Cp is used to calculate the fold change or relative difference between two sets of data. Because each cycle represents a doubling of the amplicon, the difference in 1 cycle equals a 2-fold difference in copies. Therefore, a calculation for fold change would be 2̂deltaCp.

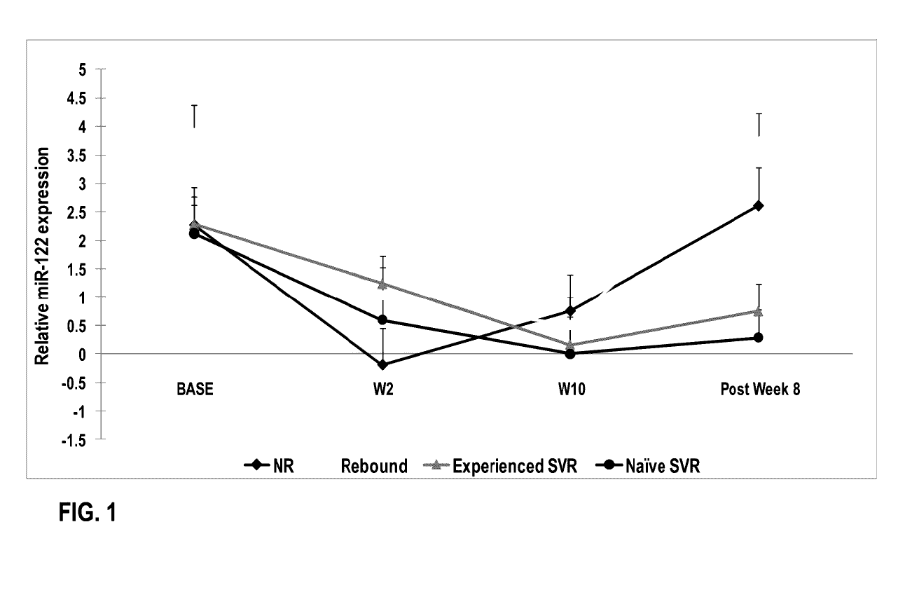

Results. As shown in FIG. 1, the patients were categorized as (1) non-responders to the direct-acting antiviral regimen (“NR”; diamonds); (2) patients experiencing a viral rebound/relapse after treatment with the direct-acting antiviral regimen (“Rebound”; squares); (3) patients who had previously been exposed to an interferon-containing treatment regimen and ultimately achieved SVR after treatment with the direct-acting antiviral regimen (“Experienced SVR”; triangles); and (4) patients who had not been treated previously and achieved SVR after treatment with the direct-acting antiviral regimen (“Naïve SVR”; circles). Levels of miR-122 were reduced in all subjects at week 2. In both Naïve SVR and Experienced SVR subjects, levels of miR-122 showed on average a 4-fold reduction between baseline and week 2, and remained at that level at all other timepoints. In contrast, in NR subjects, levels of miR-122 were similarly reduced at week 2 but increased between week 2 and week 10 and, ultimately, returned to baseline levels by post-treatment week 8.

As also shown in FIG. 1, Rebound subjects had on average a 4-fold higher baseline level of miR-122 relative to other individuals. In the Rebound subjects, the miR-122 levels were reduced following treatment, but returned to baseline between week 10 and post-treatment week 8.

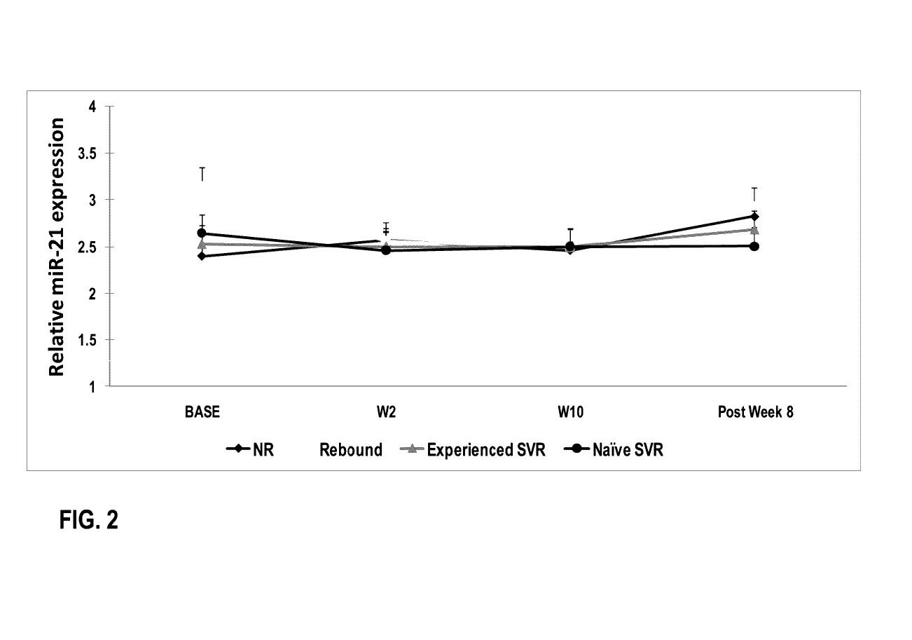

As shown in FIG. 2, the patients were categorized as (1) non-responders to the direct-acting antiviral regimen (“NR”; diamonds); (2) patients experiencing a viral rebound/relapse after treatment with the direct-acting antiviral regimen (“Rebound”; squares); (3) patients who had previously been exposed to an interferon-containing treatment regimen and ultimately achieved SVR after treatment with the direct-acting antiviral regimen (“Experienced SVR”; triangles); and (4) patients who had not been treated previously and achieved SVR after treatment with the direct-acting antiviral regimen (“Naïve SVR”; circles). Similar to miR-122, baseline serum levels of miR-21 were on average 4-fold higher in Rebound subjects relative to other individuals.

This is the first report of expression levels of circulating microRNA levels in subjects treated with an IFN-free treatment regimen for chronic HCV.

Baseline serum expression levels of both miR-122 and miR-21 were unexpectedly higher in subjects who ultimately relapsed following treatment with Compound 1/r dosed in combination with Compound 2 and RBV relative to patients who achieved SVR12 following treatment with Compound 1/r dosed in combination with Compound 2 and RBV.

In addition, the results showed that miR-122 expression levels were reduced in SVR-achieving subjects treated with Compound 1/r dosed in combination with Compound 2 and RBV. However, miR-122 expression levels returned to baseline in subjects who failed treatment with this direct-acting antiviral regimen.

These results suggest that microRNA levels can be used as biomarkers for predicting and evaluating treatment response. For example, miR-122 and/or miR-21 could be used to identifying subjects likely to achieve SVR on a direct-acting antiviral regimen.

### EXAMPLE 2

### Use of Direct-Acting Antiviral Regimen Alone and in Combination With Peginterferon α-2a and Ribavirin (PegIFN/RBV) to Treat Treatment-Naïve Subjects Infected with HCV Genotype 1

Group A. Previously untreated subjects having HCV infection were treated with a polymerase inhibitor for three days. The first three days of treatment were without interferon and without ribavirin. Following three days of monotherapy with the polymerase inhibitor, the subjects received the polymerase inhibitor at the same dose in combination with pegylated interferon/ribavirin (“P/R”) through week 12. At week 12, treatment with the polymerase inhibitor was discontinued and subjects received P/R alone through week 48. Within Group B, subjects received either Compound 4 (100 or 300 or 600 mg QD) or Compound 2 (400 mg BID). Subjects included treatment-naïve subjects between the ages of 18 and 65.

Group B. Previously untreated subjects having HCV infection were treated with P/R alone for 48 weeks (“SOC”). Subjects included treatment-naïve subjects between the ages of 18 and 65.

Expression profiling of circulating microRNA in serum was performed to identify baseline microRNA levels and changes in microRNA that occur as a result of treatment. Sample preparation and microRNA real-time PCR was performed as described for Example 1. Serum samples were analyzed from day 1 (baseline), day 3 (for response to monotherapy), week 4, and week 10.

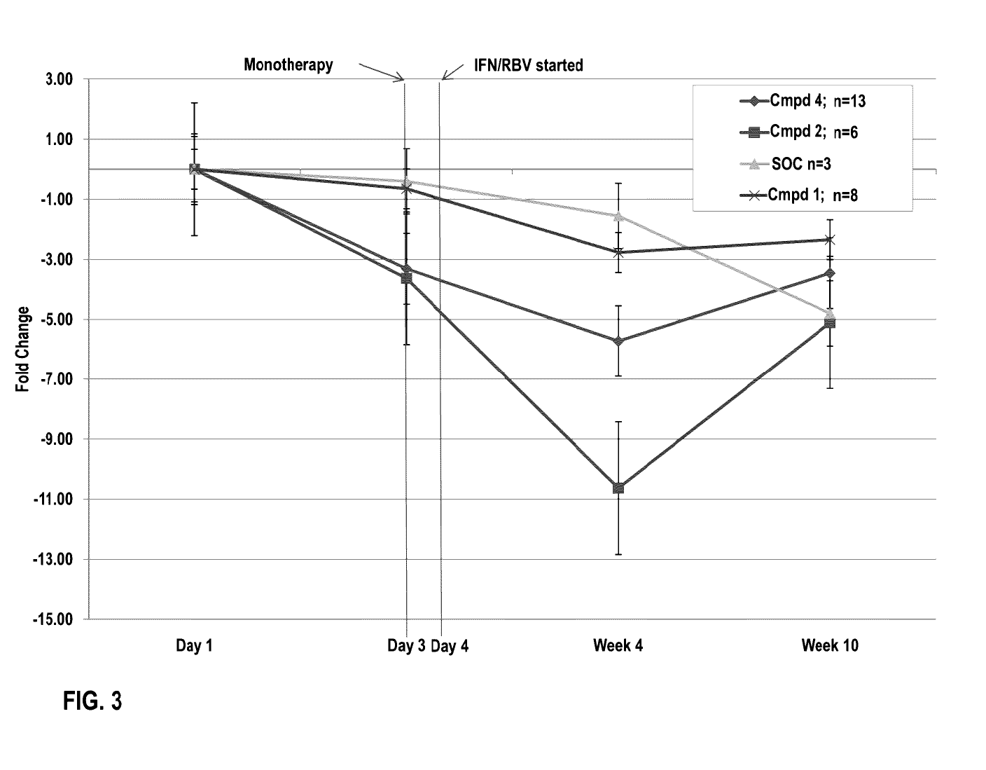

Results. Data from subjects that achieved SVR are shown in FIG. 3. The patients were categorized by treatment regimen: (1) Group A; Compound 2 (squares); (2) Group A; Compound 4 (diamonds); and (3) Group B; SOC (triangles).

As shown in FIG. 3, subjects that achieved SVR also exhibited reduced serum miR-122 levels following 10 weeks of treatment as described above. Levels of miR-122 were decreased in subjects treated with a polymerase inhibitor alone (either Compound 2 or Compound 4) for three days.

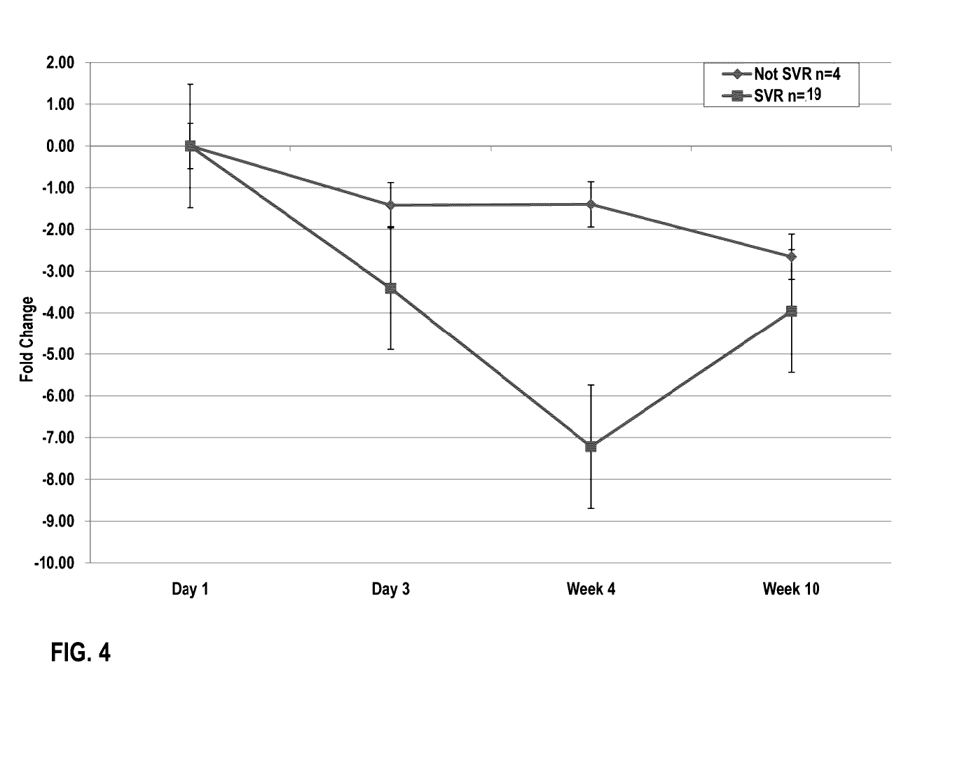

Data from subjects treated with a polymerase inhibitor (Group B) are shown in FIG. 4. The patients were categorized as achieving SVR (squares; n=19) or not achieving SVR (diamonds; n=4).

As shown in FIG. 4, subjects achieving SVR following treatment with a polymerase inhibitor exhibited a greater than 3-fold decrease in serum miR-122 levels following three days of monotherapy with the polymerase inhibitor. Moreover, at week 4, subjects achieving SVR following treatment with a polymerase inhibitor exhibited a greater than 7-fold decrease in serum miR-122 levels. In contrast, subjects who failed to achieve SVR following treatment with a polymerase inhibitor only exhibited a one- to two-fold decrease in serum miR-122 levels at day 3 and week 4 of treatment.

Serum microRNA expression levels were unexpectedly responsive to treatment with a polymerase inhibitor. Three days of monotherapy with either Compound 2 or Compound 4 produced a greater than 3-fold reduction in serum miR-122 expression levels.

In addition, the results showed that miR-122 expression levels were more dramatically decreased in SVR-achieving subjects treated with a polymerase inhibitor as opposed to non-SVR subjects.

These results suggest that microRNA levels, and miR-122 levels in particular, can be used as biomarkers for predicting and evaluating treatment response to a polymerase inhibitor. In particular, miR-122 levels can be used as biomarkers for predicting and evaluating treatment response to a non-nucleoside polymerase inhibitor, such as Compound 2 or Compound 4. miR-122 could be used to identifying subjects likely to achieve SVR on a direct-acting antiviral regimen that includes a polymerase inhibitor, and, in particular, a non-nucleoside polymerase inhibitor.

### EXAMPLE 3

### Use of Direct-Acting Antiviral Regimen Alone and in Combination With Ribavirin (RBV) to Treat Treatment-Naïve Subjects Infected with HCV Genotype 2 or 3

Genotype 2 Cohort. Previously untreated subjects having HCV genotype 2 infection were treated with a protease inhibitor (in combination with ritonavir) and an NS5A inhibitor with or without RBV. The treatment was without interferon.

Subjects included 20 treatment naïve subjects between the ages of 18 and 65. Nine patients completed 12 weeks of therapy with a direct-acting antiviral regimen comprising Compound 1/r dosed in combination with Compound 3 and RBV. Eight patients completed 12 weeks of therapy with a direct-acting antiviral regimen comprising Compound 1/r dosed in combination with Compound 3. Compound 1/r was dosed 200/100 mg QD. Compound 3 was dosed 25 mg QD. RBV was dosed 1000-1200 mg daily divided BID, based on weight. Two subjects experienced viral breakthrough and two subjects relapse following treatment.

The characteristics of the patients prior to initiating the direct-acting antiviral regimen are shown in the table below.

| | Genotype 2 |
|---|---|
| | Cohort |
| Subtype (2a/2b) | 16/4 |
| IL28B: | |
| CC | 6 |
| CT | 9 |
| TT | 5 |

Expression profiling of circulating microRNA in serum was performed to identify baseline microRNA levels as well as changes in microRNA that occur as a result of treatment in subjects infected with HCV genotype 2.

Sample preparation and microRNA real-time PCR. Serum samples were collected at baseline (“Baseline”), day 3 on-treatment (“Day 3”), week 2 on-treatment (“Week 2”), week 10 on-treatment (“Week 10”), and 8 weeks after completing the 12 week regimen (“PT Week 8”) and frozen until analysis. Samples were processed as described in Example 1.

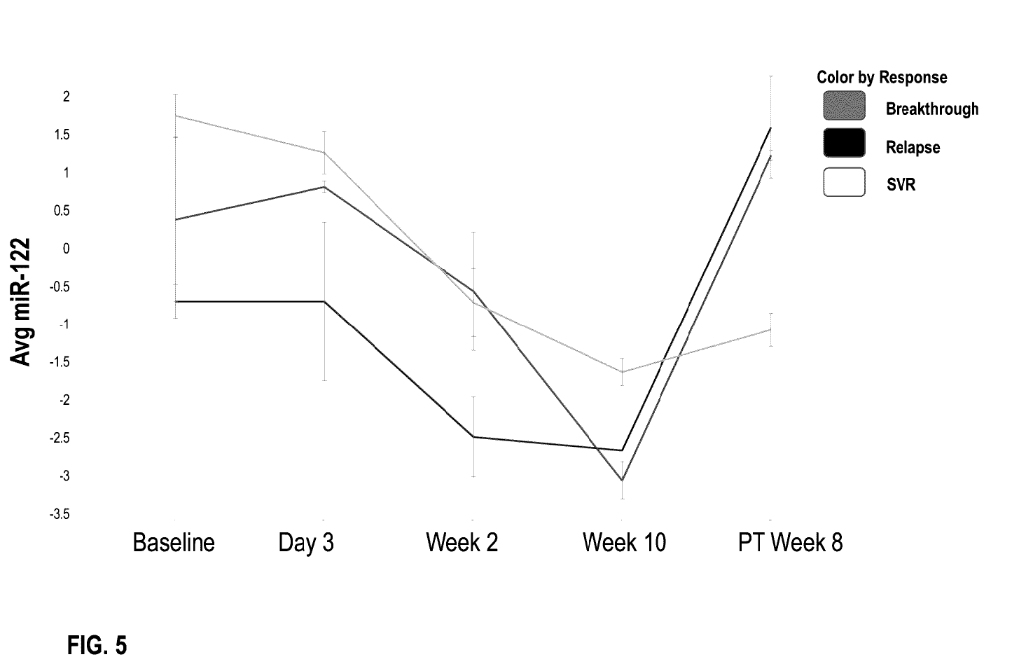

Results. As shown in FIG. 5, the patients were categorized as (1) patients experiencing viral breakthrough while on-treatment with the direct-acting antiviral regimen (“Breakthrough”); (2) patients experiencing a viral rebound/relapse after treatment with the direct-acting antiviral regimen (“Relapse”); and (3) patients achieving SVR after treatment with the direct-acting antiviral regimen (“SVR”)

In all subjects, miR-122 levels are initially decreased in the on-treatment period. miR-122 levels remain below baseline at PT-week 8 in subjects who achieve SVR. In contrast, miR-122 levels return to baseline in subjects who experience viral breakthrough (n=2) or relapse (n=2).

The results showed that miR-122 expression levels were reduced in SVR-achieving HCV genotype 2 subjects treated with Compound 1/r dosed in combination with Compound 3 with or without RBV. However, miR-122 expression levels returned to baseline in subjects who failed treatment with this direct-acting antiviral regimen.

Thus, HCV genotype 2 subjects exhibit reduced serum levels of miR-122 following treatment with an IFN-free, direct-acting antiviral regimen. Moreover, serum levels of miR-122 correlate with HCV RNA levels in HCV genotype 2 subjects.

These results suggest that microRNA levels can be used as biomarkers for evaluating treatment response in subjects infected with HCV genotype 2.

Genotype 3 Cohort. Previously untreated subjects having HCV genotype 3 infection were treated with a protease inhibitor (in combination with ritonavir) and an NS5A inhibitor with or without RBV. The treatment was without interferon.

Subjects included 21 treatment naïve subjects between the ages of 18 and 65. Eight patients completed 12 weeks of therapy with a direct-acting antiviral regimen comprising Compound 1/r dosed in combination with Compound 3 and RBV. Two patients completed 12 weeks of therapy with a direct-acting antiviral regimen comprising Compound 1/r dosed in combination with Compound 3. Compound 1/r was dosed 200/100 mg QD. Compound 3 was dosed 25 mg QD. RBV was dosed 1000-1200 mg daily divided BID, based on weight. Eleven subjects experienced viral breakthrough and two subjects relapse following treatment.

The characteristics of the patients prior to initiating the direct-acting antiviral regimen are shown in the table below.

| | Genotype 3 |
|---|---|
| IL28B: | Cohort |
| CC | 5 |
| CT | 14 |
| TT | 2 |

Expression profiling of circulating microRNA in serum was performed to identify baseline microRNA levels as well as changes in microRNA that occur as a result of treatment in subjects infected with HCV genotype 3.

Sample preparation and microRNA real-time PCR. Serum samples were collected at baseline (“Baseline”), day 3 on-treatment (“Day 3”), week 2 on-treatment (“Week 2”), week 10 on-treatment (“Week 10”), and 8 weeks after completing the 12 week regimen (“PT Week 8”) and frozen until analysis. Samples were processed as described in Example 1.

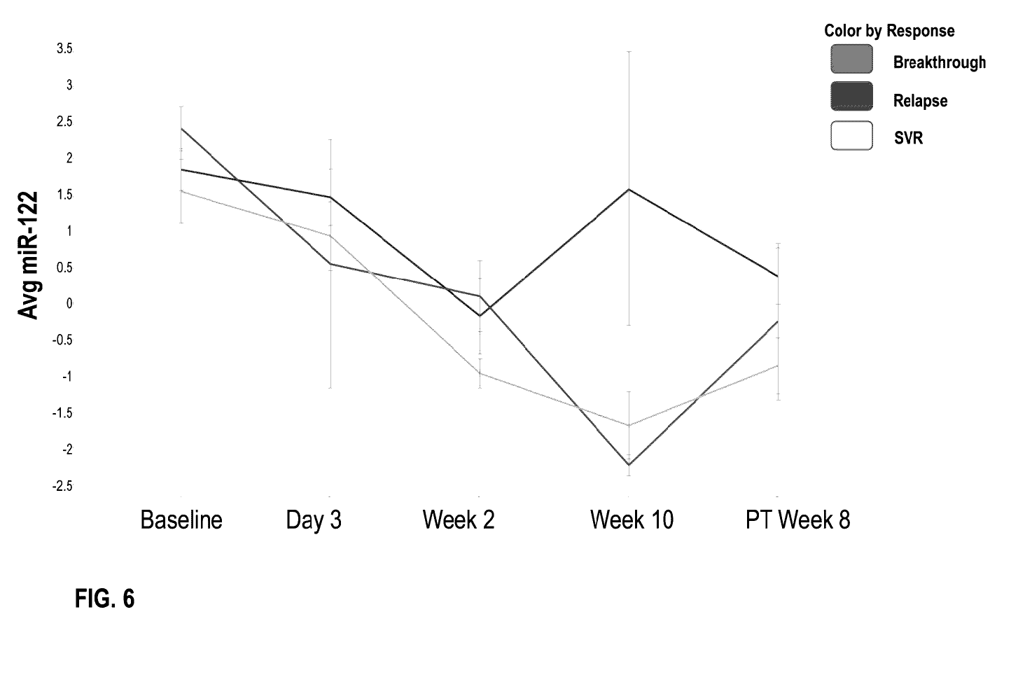

Results. As shown in FIG. 6, the patients were categorized as (1) patients experiencing viral breakthrough while on-treatment with the direct-acting antiviral regimen (“Breakthrough”); (2) patients experiencing a viral rebound/relapse after treatment with the direct-acting antiviral regimen (“Relapse”); and (3) patients achieving SVR after treatment with the direct-acting antiviral regimen (“SVR”).

In all subjects, miR-122 levels are initially decreased in the on-treatment period. miR-122 levels remain below baseline at PT-week 8 in subjects who achieve SVR. In contrast, miR-122 levels return to baseline in subjects who experience viral breakthrough and increase toward baseline in subjects who relapse (n=2).

The results showed that miR-122 expression levels were reduced in SVR-achieving HCV genotype 3 subjects treated with Compound 1/r dosed in combination with Compound 3 with or without RBV. However, miR-122 expression levels returned to baseline in subjects who failed treatment with this direct-acting antiviral regimen.

Thus, HCV genotype 3 subjects exhibit reduced serum levels of miR-122 following treatment with an IFN-free, direct-acting antiviral regimen. Moreover, serum levels of miR-122 correlate with HCV RNA levels in HCV genotype 3 subjects.

These results suggest that microRNA levels can be used as biomarkers for evaluating treatment response in subjects infected with HCV genotype 3.

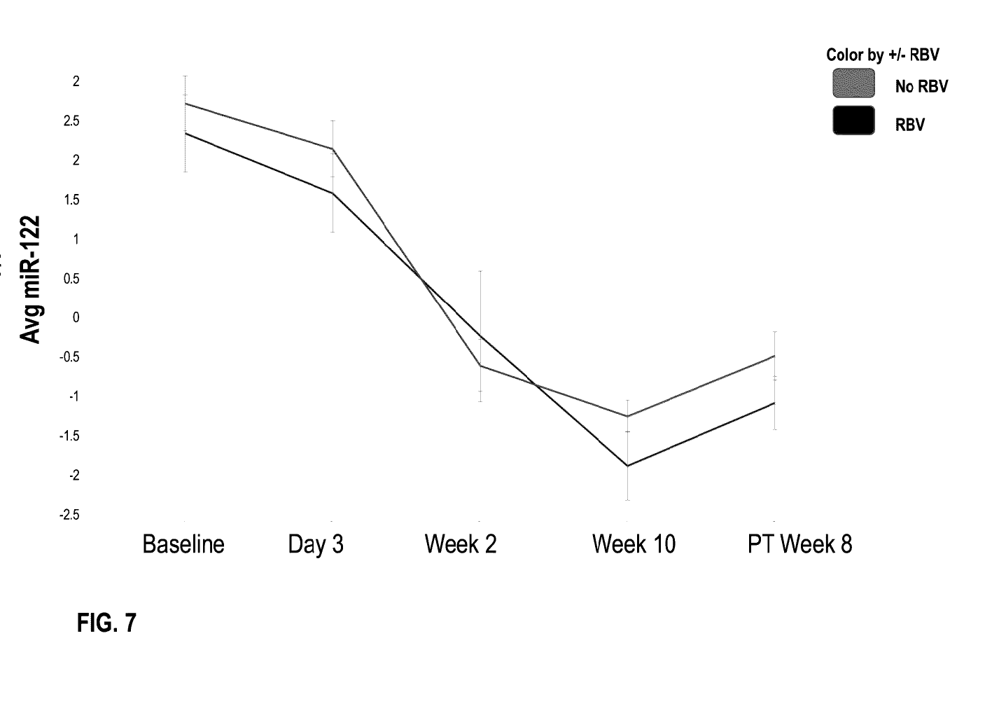

FIG. 7 shows miR-122 levels in genotype 2 and genotype 3 patients that achieved SVR following treatment with a direct-acting antiviral regimen comprising Compound 1/r dosed in combination with Compound 3 with or without RBV. As shown in FIG. 7, the patients were categorized as (1) receiving the direct-acting antiviral regimen containing RBV; and (2) receiving the direct-acting antiviral regimen without RBV. In HCV 2 and 3 subjects who achieve SVR, miR-122 levels are not affected by the presence (n=13) or absence (n=9) of RBV.

The foregoing description of the present invention provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

What is claimed is:

1. A method to treat a patient infected with Hepatitis C virus (HCV) genotype 2 or HCV genotype 3 with a direct-acting antiviral regimen, the method comprising:
administering the direct-acting antiviral regimen to the patient;
wherein prior to the administration of the direct-acting antiviral regimen a blood sample from the patient has been tested to determine a baseline miR-122 level.

2. The method of claim 1, wherein the baseline miR-122 level is significantly less than a mean miR-122 level in a population of HCV patients who fail to achieve a sustained virological response following treatment with the direct-acting antiviral regimen

3. The method of claim 1, wherein the blood sample is a plasma sample.

4. The method of claim 1, wherein the blood sample is a serum sample.

5. The method of claim 1, wherein the direct-acting antiviral regimen comprises an HCV protease inhibitor and an HCV polymerase inhibitor.

6. The method of claim 5, wherein the HCV protease inhibitor is therapeutic agent 1 and the HCV polymerase inhibitor is therapeutic agent 2.

7. The method of claim 1, further comprising monitoring miR-122 levels in the patient at or between week 2 and week 10 after commencing administration of the direct-acting antiviral regimen.

8. The method of claim 1, wherein the patient is infected with HCV genotype 2.

9. The method of claim 1, wherein the patient is infected with HCV genotype 3.

10. A method to predict responsiveness of a patient infected with Hepatitis C virus (HCV) genotype 2 or HCV genotype 3, comprising
(a) providing a sample from the patient;
(b) assessing microRNA expression in the sample to obtain a microRNA expression level; and
(c) predicting, based on the microRNA expression level, responsiveness to the direct-acting antiviral regimen.

11. The method of claim 10, wherein a microRNA expression level that is less than or equal to a pre-determined control level is predictive of a sustained response to the direct-acting antiviral regimen.

12. The method of claim 10, wherein a microRNA expression level that is greater than a pre-determined control level predicts an inadequate sustained response to treatment with the direct-acting antiviral regimen.

13. The method of claim 10, wherein the microRNA is miR122.

14. The method of claim 10, wherein the step of assessing microRNA expression comprises hybridizing a nucleic acid primer or probe to the microRNA or a complementary sequence thereof to form a detectable complex.

15. The method of claim 10, wherein the step of assessing microRNA expression comprises amplifying all or part of the microRNA or complementary sequence thereof.

16. The method of claim 10, wherein the step of assessing microRNA expression generating cDNA from the sample and sequencing at least a portion of the cDNA.

17. A method to treat a patient having a Hepatitis C virus (HCV) genotype 2 or HCV genotype 3 infection, the method comprising:
administering a first direct-acting antiviral regimen to the patient, wherein the first direct-acting antiviral regimen comprises a first polymerase inhibitor;
assessing miR-122 expression in a sample obtained from the patient after the administration of the first direct-acting antiviral regimen to establish an on-treatment miR-122 expression level; and
administering a second direct-acting antiviral regimen to the patient when the on-treatment miR-122 expression level is not substantially different from a baseline miR-122 expression level from a sample obtained from the patient prior to administration of the first direct-acting antiviral regimen.

18. The method of claim 17, wherein the sample obtained from the patient prior to administration of the first direct-acting antiviral regimen is a serum or plasma sample and/or wherein the sample obtained from the patient after the administration of the first direct-acting antiviral regimen or is a serum or plasma sample.

19. The method of claim 17, wherein the second direct-acting antiviral regimen comprises an increased dose of the first polymerase inhibitor and/or a second polymerase inhibitor.

20. The method of claim 17, wherein the sample obtained from the patient after the administration of the first direct-acting antiviral regimen is obtained at or between week 2 and week 10 after commencing administration of the first direct-acting antiviral regimen.
